SEQUENCE LISTING (12 sequences)

SEQ ID NO: 1 - protein, 25 aa, synthetic construct
YQDMVLWKDVFRKNNQLAPVGPSLC

SEQ ID NO: 2 - protein, 19 aa, synthetic construct
CGGGVFLQKYPHTHLVHQA

SEQ ID NO: 3 - protein, 19 aa, synthetic construct
CGGGFASTEKSNIIRGWIF

SEQ ID NO: 4 - protein, 19 aa, synthetic construct
CGGGPFLGVYYHKNNKSWM

SEQ ID NO: 5 - protein, 20 aa, synthetic construct
CGGGNNLDSKVGGNYNYLYR

SEQ ID NO: 6 - protein, 22 aa, synthetic construct
CGGGPSKPSKRSFIEDLLFNKV

SEQ ID NO: 7 - protein, 35 aa, synthetic construct
KDEPQRRSARLSAKPAPPKPEPKPKKAPAKKGGGC

SEQ ID NO: 8 - protein, 17 aa, synthetic construct
CSGGGWGWSLSHGYQVK

SEQ ID NO: 9 - protein, 23 aa, synthetic construct
CSGGGDWFKAFYDKVAEKFKEAF

SEQ ID NO: 10 - protein, 4 aa, synthetic construct
GGSC

SEQ ID NO: 11 - protein, 4 aa, synthetic construct
GGGC

SEQ ID NO: 12 - protein, 5 aa, synthetic construct
GGGSC

(19) United States
(12) Patent Application Publication
(10) Pub. No.: US 2024/0350422 A1
(43) Pub. Date: Oct. 24, 2024

(54) FORMULATION PROCESS FOR CPMV NANOPARTICLES

(71) Applicant: The Regents of the University of California, Oakland, CA (US)

(72) Inventors: Nicole F. Steinmetz, La Jolla, CA (US); Matthew D. Shin, La Jolla, CA (US)

(21) Appl. No.: 18/580,090

(22) PCT Filed: Jul. 25, 2022
(86) PCT No.: PCT/US2022/038227

§ 371 Date: Jan. 17, 2024

Related U.S. Application Data
(60) Provisional application No. 63/225,856, filed on Jul. 26, 2021.

(51) Int. Cl. A61K 9/51; A61K 35/76

(52) U.S. Cl. CPC A61K 9/5184; A61K 9/5192; A61K 35/76

(57) ABSTRACT

Proteinaceous nanoparticles can be used to deliver large payloads of active ingredients, which is advantageous in medicine and agriculture. However, the conjugation of hydrophobic ligands to hydrophilic nanocarriers such as plant viral nanoparticles (plant VNPs) can result in aggregation by reducing overall solubility. Provided herein are improved methods for ligand conjugations, as well as the particles prepared by these methods. The disclosed methods promote efficient bioconjugation and prevent the aggregation that accompanies conventional bioconjugation methods, thus improving the stability, homogeneity, and translational potential of plant VNP conjugates in medicine and agriculture.

### CROSS-REFERENCE TO RELATED APPLICATIONS

This application claims priority under 35 U.S.C. § 119 (e) to U.S. Provisional Application No. 63/225,856, filed Jul. 26, 2021, the contents of which are incorporated herein by reference in their entireties.

### STATEMENT OF GOVERNMENT SUPPORT

This invention was made with government support under CA202814 and HL137674 awarded by the National Institutes of Health (NIH), under CMMI2027668 and DMR2011924 awarded by the National Science Foundation (NSF), and under 20206702131255 awarded by the United States Department of Agriculture (USDA). The government has certain rights in the invention.

### BACKGROUND

The following discussion of the background is merely provided to aid the reader in the understanding the disclosure and is not admitted to describe or constitute prior art to the present disclosure. Throughout and within this disclosure, various patent and technical publications are referenced by an identifying citation or an Arabic number, the full bibliographic citation for which can be found herein. These disclosure are incorporated herein to more fully describe the state of the art to which this disclosure pertains.

Nanocarrier display of therapeutic, targeting, and imaging active ingredients (a.i.) of hydrophobic nature are desirable due to the inherent benefits of nanoparticles which can enable controlled and sustaineddrug release, multivalent ligand display/high payload delivery to target diseased tissues or organs. Importantly, nanoformulations afford improved solubility of hydrophobic a.i. in aqueous medium. Multivalency also holds merit in vaccine design as the number, spacing, and avidity of ligands can be immunoengineered to modulate B-cell activation.^{[4]} Payload display can be achieved by genetic or chemical bioconjugation; the former of course only when the target moiety is a biologic that can be expressed from a gene. Bioconjugate chemistry applied to any a.i. payload (biological or not) offers speed, albeit greater batch to batch variability-bioconjugation reactions are typically carried out in the order of hours while genetic engineering approaches generally require several days.

### SUMMARY OF THE DISCLOSURE

Thus, in one aspect, provided herein is a formulation process comprising: a) admixing a viral nanoparticle (VNP) with an effective amount of about 4 wt % of a poloxamer (e.g. Pluronic F127 (F127)) or an equivalent thereof to prepare a first mixture at a temperature of about 25° C.; b) cooling the mixture conjugate at a temperature below about 19.5° C. to promote the poloxamers such as F127 unimerization and then returning the mixture to a temperature of about 25° C. to promote the poloxamer such as F127 micellization; and c) mixing the mixture prepared by step b). In one aspect, steps b) and c) are repeated at least twice or at least thrice. In one aspect, the VNP is selected from: tobacco mosaic virus (TMV), cowpea mosaic virus (CPMV) or tobacco mild green mosaic virus (TMGMV), and a modified equivalent thereof. Non-liming examples of modified equivalents include TMV-lysine, TMV-lysine with a Lys substitution at amino acid position 158, CPMV-lysine, CPMV- with a Lys substitution at amino acid position 158, chemically conjugated TMV, chemically conjugated CPMV, chemically conjugated TMGMV, TMV-Cys5.5, ligand-modified TMV, ligand-modified CPMV, ligand-modified TMGMV, avermectin-TMGMV or TMGMV-Cys5. In another aspect, the ligand-modified TMV, ligand-modified CPMV, or ligand-modified TMGMV is modified with a ligand selected from a peptide epitope for a vaccine, a peptide epitope for a COVID vaccine, a targeting peptide, an agrochemical, a detectable label, a small molecule, a fluorophore, or a peptide of Table 1. In another aspect, the ligand is hydrophobic. In a yet further aspect, the ligand is a peptide that comprises a Cys-terminal GGSC or GGGC linker.

In one embodiment, the method is performed with from about 15 to about 45 molar equivalents of a poloxamer such as F127 or an equivalent per coat protein (CP) of the VNP or about 28 molar equivalents of a poloxamer such as F127 or an equivalent per CP.

In a further aspect, in step a) of the method, the VNP and a poloxamer such as F127 or an equivalent are mixed with a hydrophobic Cy5.5-NHS ligand at about 2 molar equivalents per CP, and optionally wherein steps b) and c) are omitted. In one aspect, the VNP comprises TMV.

In a further aspect of the method, in step a), the VNP comprises a TMGMV-ds and a poloxamer such as F127 are mixed with avermectin ligand at about 5 molar equivalents per CP, in the presence of Cu(I)-catalyzed azide-alkyne cycloaddition, and optionally wherein steps b) and c) are omitted.

In a further aspect of the method, in step a), the VNP comprises a maleimide-bearing VNP-M(PEG)₄.

In a yet further aspect of the method, the VNP comprises maleimide-bearing M(PEG)₄TMV or maleimide-bearing M(PEG)₄CPMV.

In another embodiment of the method in step a), the VNP comprises a maleimide-bearing CPMV-M(PEG)₄ mixed with F127 or an equivalent thereof and a peptide. Non-limiting examples of the peptides include a COVID19 epitope, a HER2 epitope, cyclosporine A peptide, MyoI, or a peptide of Table 1.

Further provided is a process for a hydrophobic peptide comprising: a) dialyzing a water-insoluble peptide dissolved in DMSO and about 10 wt % of a poloxamer such as F127 or an equivalent thereof to form a peptide-loaded micelle (a FORM peptides); b) mixing the FORM peptide with an M(PEG)₄-VNP with an effective amount of about 4 wt % of a poloxamer such as Pluronic F127 (F127) or an equivalent thereof to prepare a first mixture at a temperature of about 25° C.; c) cooling the mixture conjugate at a temperature below about 19.5° C. to promote the poloxamer such as F127 unimerization and then returning the mixture to a temperature of about 25° C. to promote the poloxamer such as F127 micellization; and d) mixing the mixture prepared by step c). In one aspect of the method, from about 15 to about 45 molar equivalents of the poloxamer such as F127 or an equivalent per coat protein (CP) of the VNP or about 28 molar equivalents of the poloxamer such as F127 or an equivalent per CP are mixed.

In one aspect of this method, steps c) and d) are repeated at least twice or at least thrice.

In several aspects of the above methods, the VNP or the a.i. can be detectably labeled. “Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein.

Also provided are the formulations prepared by the method as disclosed herein.

Further provided are compositions comprising the formulations and a carrier. In one aspect, the carrier is a pharmaceutically acceptable carrier. Non-limiting examples of pharmaceutically acceptable carriers any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

Yet further provided is a delivery method comprising administering to a subject in need thereof the formulation as disclosed herein or a composition comprising the formulation. In one aspect, the subject is a mammal or a human. Administration can be effected in one dose, continuously or intermittently throughout the course of treatment. Methods of determining the most effective means and dosage of administration are known to those of skill in the art and will vary with the composition used for therapy, the purpose of the therapy, as well as the age, health or gender of the subject being treated. Single or multiple administrations can be carried out with the dose level and pattern being selected by the treating physician or in the case of pets and animals, treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated and the target cell or tissue. Non-limiting examples of route of administration include intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, and inhalation.

### BRIEF DESCRIPTION OF THE DRAWINGS

FIG. 1: FIG. 1 illustrates an exemplary COAT strategy. Plant VNPs are mixed with 4% (w/w) F127 and then processed by varying the temperature. The samples are first incubated on ice to promote the formation of F127 unimers before shifting to room temperature (RT) to promote F127 micellization and subsequent plant VNP coating.

FIGS. 2A-2C: DLS data showing plant VNP size following the COAT process and fixed-temperature ultracentrifugation. (FIG. 2A) Plant VNPs mixed with F127 using the COAT strategy at 19.5° C. (FIG. 2B) Coated plant VNPs are purified by ultracentrifugation at 25° C. (FIG. 2C) Coated plant VNPs are purified by ultracentrifugation at 4° C. Top row=TMV, bottom row=CPMV.

FIG. 3A-3B: Summary of plant VNP bioconjugation with Cy5.5 and avermectin using the COAT strategy. FIG. 3A shows an exemplary synthesis of Cy5.5-conjugated TMV via NHS ester coupling. FIG. 3B shows an exemplary synthesis of avermectin-conjugated TMGMV via Cu-catalyzed click chemistry.

FIGS. 4A-4E: Chemical conjugation of Cy5.5 dye to TMV using amine-reactive NHS chemistry. (FIG. 4A) SDS-PAGE analysis of TMV-Cy5.5 particles using MultiFluor Red (632 nm) and Coomassie Brilliant Blue and the Fluorchem M System. CP=TMV coat protein (˜17 kDa). (FIG. 4B) Photograph of TMV-Cy5.5 particles with/without (+/−) the COAT pre-incubation strategy, showing aggregation in the sample without pre-incubation. (FIG. 4C) Corresponding DLS intensities of TMV particles (Zₐᵥ data are mean values±SD, n=3). (FIG. 4D) Corresponding TEM images of negatively-stained TMV particles. (FIG. 4E) UV/Vis absorbance of native TMV and Cy5-conjugated to TMV (+/−F127) after a 30-min reaction.

FIGS. 5A-5D: Chemical conjugation of Avermectin azide (Avm) to TMGMV using Cu-catalyzed click chemistry. (FIG. 5A) Photograph of TMGMV-Avm particles with/without (+/−) the COAT pre-incubation strategy, showing aggregation in the sample without pre-incubation. (FIG. 5B) UV/Vis absorbance of native TMGMV, Cy5-conjugated TMGMV, and Cy5-conjugated TMGMV-Avm(+/−F127). The left panel shows groups of TMGMV-Avm conjugates after 3 or 24 h, and the right panel shows a group of TMGMV-Avm conjugates after 30 min. Number in parentheses is the number of Avm ligands per TMGMV. (FIG. 5C) Corresponding DLS intensities of TMGMV particles (Zₐᵥ data are mean values±SD, n=3). (FIG. 5D) Corresponding TEM images of negatively-stained TMGMV particles.

FIGS. 6A-6B: Show an exemplary synthesis of peptide-conjugated TMV and CPMV particles using a dual-functional M(PEG)₄ linker with maleimide-thiol and NHS-amine chemistry. In this aspect, the COAT strategy was applied to the intermediate plant VNP-SM(PEG)₄ before peptide bioconjugation.

FIGS. 7A-7D: Chemical conjugation of peptide ScG3 via an SM(PEG)₄ linker to TMV, which is also labeled internally with Cy5. (FIG. 7A) SDS-PAGE analysis of TMV-ScG3 particles. CP=TMV coat protein (˜17 kDa). (FIG. 7B) Photograph of TMV-ScG3 particles with/without (+/−) the COAT pre-incubation strategy, showing aggregation in the sample without pre-incubation. (FIG. 7C) Corresponding DLS intensities of TMV particles (Zₐᵥ data are mean values±SD, n=3). (FIG. 7D) Corresponding TEM images of negatively-stained TMV particles.

FIGS. 8A-8D: Chemical conjugation of peptide F3 via an SM(PEG)₄ linker to TMV. (FIG. 8A) SDS-PAGE analysis of TMV-F3 particles. CP=TMV coat protein (˜17 kDa). (FIG. 8B) Photograph of TMV-F3 particles with/without (+/−) the COAT pre-incubation strategy, showing aggregation in the sample without pre-incubation. (FIG. 8C) Corresponding DLS intensities of TMV particles (Zₐᵥ data are mean values±SD, n=3). (FIG. 8D) Corresponding TEM images of negatively-stained TMV particles.

FIGS. 9A-9D: Chemical conjugation of peptide ApoAI via an SM(PEG)₄ linker to CPMV. (FIG. 9A) SDS-PAGE analysis of CPMV-ApoAI particles. S-CP=CPMV small coat protein (˜24 kDa) and L-CP=CPMV large coat protein (˜42 kDa). (FIG. 9B) Photograph of CPMV-ApoAI particles with/without (+/−) the COAT pre-incubation strategy, showing aggregation in the sample without pre-incubation. (FIG. 9C) Corresponding TEM images of negatively-stained CPMV particles. (FIG. 9D) Corresponding DLS intensities of TMV particles (Zₐᵥ data are mean values±SD, n=3).

FIGS. 10A-10F: Chemical conjugation of peptides MyoI and COVID-826 via maleimide-thiol conjugation to CPMV. (FIG. 10A) SDS-PAGE analysis of CPMV-MyoI particles. S-CP=CPMV small coat protein (˜24 kDa) and L-CP=CPMV large coat protein (˜42 kDa). (FIG. 10B) Agarose gel electrophoresis of CPMV-MyoI particles with/without (+/−) the COAT pre-incubation strategy. (FIG. 10C) SDS-PAGE analysis of CPMV-826 particles. (FIG. 10D) Photograph of CPMV-MyoI and CPMV-826 particles with/without (+/−) the COAT pre-incubation strategy, showing aggregation in the samples without pre-incubation. (FIG. 10E) Corresponding TEM images of negatively-stained CPMV particles. (FIG. 10F) Corresponding DLS intensities of CPMV-MyoI and CPMV-826 particles (Zₐᵥ data are mean values±SD, n=3).

FIG. 11: FIG. 11 illustrates an exemplary procedure for the formulation of hydrophobic peptides. Each peptide was first dissolved in DMSO to a concentration of 10 mg/mL before mixing with 10% (w/w) F127. The mixture was then transferred to dialysis tubing that allows solvent exchange while retaining the peptide. As DMSO leaves the dialysis bag, the F127 forms micelles and traps the hydrophobic peptides within the micelle corona. The now micellized, water-soluble peptide (formulated or FORM peptide) is collected for reaction with COATed plant VNPs in the FORMCOAT procedure.

FIG. 12: FIG. 12 shows an exemplary method to conjugate peptides to CPMV via an SM(PEG)₄ linker using the FORMCOAT strategy. Formulated peptides are combined with plant VNP-M(PEG)₄ that has already been coated with F127. This maintains the hydrophobic peptides in a soluble form and facilitates conjugation.

FIGS. 13A-13H: Chemical conjugation of peptides COVID-106, COVID-153, COVID-454 and CH401R to CPMV using maleimide-thiol chemistry. (FIG. 13A) SDS-PAGE analysis of CPMV conjugates to SARS-COV-2 peptides. S-CP=CPMV small coat protein (˜24 kDa) and L-CP=CPMV large coat protein (˜42 kDa). (FIG. 13B) Characterization of CPMV-CH401R particles by SDS-PAGE and agarose gel electrophoresis. (FIG. 13C) Densitometric comparison of conjugates produced using the FORMCOAT strategy and conventional bioconjugation. (FIG. 13D) Photograph of CPMV-peptide particles with/without (+/−) the FORMCOAT pre-incubation strategy, showing aggregation in the sample without pre-incubation. (FIG. 13E) Photograph of CPMV-peptide particles prepared with and without the prior formulation of peptides in F127 micelles, showing aggregation for the samples without prior peptide formulation. (FIGS. 13F-13G) Corresponding DLS intensities of CPMV-peptide particles (Zₐᵥ data are mean values±SD, n=3). (FIG. 13H) Corresponding TEM images of negatively-stained CPMV particles.

FIG. 14: FIG. 14 is an illustrative summary of an embodiment of this disclosure.

FIG. 15: FIG. 15 is an illustrative summary of an embodiment of this disclosure and in particular, an embodiment involving Avermectin synthesis.

FIGS. 16A-16B: ESI-MS spectra of unmodified Avermectin (FIG. 16A) and avermectin azide (FIG. 16B).

FIGS. 17A-17B: Analysis of plant VNPs by FT-IR spectroscopy following F127 coating (COAT strategy) and cold-temperature ultracentrifugation. (FIG. 17A) CPMV FT-IR region indicated by the boxed area highlights the signal retention in purified CPMV (˜2800-2900 cm⁻¹), which is a characteristic C—H stretching vibration of F12753; (FIG. 17B) TMV FT-IR region indicated by the boxed region highlights a shift in peak from 1750 cm⁻¹ toward 1650 cm⁻¹. The disappearance of the characteristic peak of purified TMV at 1750 cm⁻¹ indicates supramolecular interactions pertaining to F127 micelle coating. The region indicated by the 1100 cm⁻¹ region box highlights the signal retention in purified TMV (˜1100 cm⁻¹), which is a characteristic C—O—C stretching vibration of F127. The data suggest residual F127 present on the plant VNP surface.

FIG. 18: ELISA data showing plant VNP recognition by anti-plant VNP polyclonal antibodies. Experimental samples were purified by cold-temperature ultracentrifugation (FIG. 2C) before plating. Data (n=3) are means±SE (*p<0.05; unpaired, two-tailed Student's t-test).

FIG. 19: Superimposed intensity plots of TMV conjugates synthesized using the COAT strategy indicate a lower peak width and PDI than unconjugated TMV. This suggests that the presence of ligands corresponds to a decrease in F127 “holding power” on the surface of TMV, promoting F127 removal during low-temperature ultracentrifugation.

FIG. 20: Data plot showing the number of Cy5 ligands conjugated to TMV calculated using the Beer-Lambert law (see methods). Data (n=3) are means±SE (*p<0.001; unpaired, two-tailed Student's t-test).

FIG. 21: FIG. 21 illustrates an exemplary bioconjugation decision tree. Predicting the Log P value of a new ligand can avoid unnecessary screening experiments, saving time and resources.

### DETAILED DESCRIPTION

### Definitions

Unless defined otherwise, all technical and scientific terms used herein have the same meanings as commonly understood by one of ordinary skill in the art to which this disclosure belongs. All nucleotide sequences provided herein are presented in the 5′ to 3′ direction. All polypeptide and protein sequences are presented in the direction of the amine terminus to carboxy terminus. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. In one aspect, the term about intends variation of (+) or (−) of 0.5, or about 1.0, or about 1.5, or about 2.0, or about 2.5, or about 3.0, or about 3.5, or about 4.0, or about 4.5 or about 5.0 from the numerical designation, for example temperature. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, “and/or” refers to and encompasses any and all possible combinations of one or more of the associated listed items, as well as the lack of combinations when interpreted in the alternative (“or”).

“Optional” or “optionally” means that the subsequently described circumstance may or may not occur, so that the description includes instances where the circumstance occurs and instances where it does not.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, and pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

As used herein, the terms “treating,” “treatment” and the like are used herein to mean obtaining a desired pharmacologic and/or physiologic effect. In some embodiments, the effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, e.g., a cancer such as breast cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, longer time to tumor progression, inhibition of metastasis or a reduction in metastasis of the tumor. In one aspect, treatment excludes prophylaxis.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

As used herein, an anticancer agent refers to any drug or compound used for anticancer treatment. These include any drug that renders or maintains a clinical symptom or diagnostic marker of tumors and cancer, alone or in combination with other compounds, that reduces or maintains a state of remission, reduction, remission, prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin Kl-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives (as defined for imaging agents) thereof.

As used herein, an ablative therapy is a treatment destroying or ablating cancer tumors. In one embodiment, the ablative therapy does not require invasive surgery. In other embodiments, the ablative therapy refers to removal of a tumor via surgery. In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g. IL-2, interferon's, cytokine inducers).

In some embodiments, a method as disclosed herein further includes the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

The term “COVID” refers a strain of coronavirus that causes COVID-19 (coronavirus disease 2019), the respiratory illness responsible for the ongoing COVID-19 pandemic. It is a positive-sense single-stranded RNA (+ssRNA) virus, with a single linear RNA segment. SARS CoV 2 is a member of the subgenus Sarbecovirus (beta-CoV lineage B). Like other coronaviruses, SARS-COV-2 has four structural proteins, known as the S (spike), E (envelope), M (membrane), and N (nucleocapsid) proteins; the N protein holds the RNA genome, and the S, E, and M proteins together create the viral envelope.

The term “viral nanoparticle” (VNP) as used herein, refers to particles generally composed of one or more viral proteins, such as, but not limited to, those proteins referred to as capsid, coat, shell, surface and/or envelope proteins, or particle-forming polypeptides derived from these proteins. VNPs can form spontaneously upon recombinant expression of the protein in an appropriate expression system. VNPs can also be engineered, e.g., comprising, or consisting essentially of, or yet further consisting of, one or more viral proteins that comprise, or consists essentially of, or yet further consists of, a modification. Methods for producing VNPs are known in the art. The presence of VNPs following recombinant expression of viral proteins can be detected using conventional techniques known in the art, such as by electron microscopy, biophysical characterization, and the like. Further, VNPs can be isolated by known techniques, e.g., density gradient centrifugation and identified by characteristic density banding.

Cowpea mosaic virus (CPMV) is a plant-infecting member of the order Picornavirales, with a relatively simple, non-enveloped capsid that has been extensively studied and a positive-sense, single-stranded RNA genome. For CPMV, the genome is bipartite, with RNA-1 (6 kb) and RNA-2 (3.5 kb) being separately encapsidated. CPMV has an icosahedral capsid structure, which is ˜30 nm in diameter and is formed from 60 copies each of a Large (L) and Small(S) coat protein. These two coat proteins are processed from a single RNA-2-encoded precursor polyprotein (VP60) by the action of the 24 K viral proteinase which is encoded by RNA-1. Thus capsid assembly, as well as viral infection, is dependent on the presence of both genomic segments in an infected plant cell.

The terms “CPMV” “CPMV virus” or “CPMV particles” are used interchangeably, referring to a CPMV comprising, or alternatively consisting essentially of, or yet consisting of a capsid and an RNA genome (which is also referred to herein as a viral genome) encapsidated in the capsid. In some embodiments, the CPMV particles have been treated, prepared and/or inactivated by a method as disclosed herein. In some embodiments, the CPMV particle further comprises a heterologous RNA, which is heterologous to (i.e., not naturally presented in) a native CPMV free of any human intervention.

The virus can be obtained according to various methods known to those skilled in the art. In embodiments where plant virus particles are used, the virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393 (2): 329-37 (2009). The disclosures of both of these references are incorporated herein by reference.

As used herein, an active ingredient (a.i.) can be an anticancer agent. An anticancer agent refers to any drug or compound used for anticancer treatment. These include any drug that renders or maintains a clinical symptom or diagnostic marker of tumors and cancer, alone or in combination with other compounds that reduces or maintains a state of remission, reduction, remission, prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin Kl-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives (as defined for imaging agents) thereof.

As used herein, the term “animal” refers to living multi-cellular vertebrate organisms, a category that includes, for example, mammals and birds. The term “mammal” includes both human and non-human mammals.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

The term “PEG” as used herein refers to polyethylene glycol, a polyether compound derived from petroleum with many applications, from industrial manufacturing to medicine. PEG is also known as polyethylene oxide (PEO) or polyoxyethylene (POE), depending on its molecular weight. The structure of PEG is commonly expressed as H—(O—CH2-CH2)ₙ-OH.

Pluronic F127 is a trade name for Poloxamer 407, a hydrophilic non-ionic surfactant of the more general class of copolymers known as poloxamers. Thus, as used herein, an equivalent of Pluronic F127 includes poloxamers that are triblock copolymers consisting of a central hydrophobic block of polypropylene glycol flanked by two hydrophilic blocks of polyethylene glycol (PEG). The approximate lengths of the two PEG blocks is 101 repeat units, while the approximate length of the propylene glycol block is 56 repeat units. Pluronic F127 is commercially available from BASF. An equivalent is available from Croda under the trade name Synperonic PE/F 127.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The compositions can contain a detectable label for diagnostic or research purposes. Non-limiting exemplary detectable labels also include a radioactive material, such as a radioisotope, a metal or a metal oxide. Radioisotopes include radionuclides emitting alpha, beta or gamma radiation. In particular embodiments, a radioisotope can be one or more of: ³H, ¹⁰B, ¹⁸F, ¹¹C, ¹⁴C, ¹³N, ¹⁸O, ¹⁵O, ³²P, P³³, ³⁵S, ³⁵Cl, ⁴⁵Ti, ⁴⁶Sc, ⁴⁷Sc, ⁵¹Cr, ⁵²Fe, ⁵⁹Fe, ⁵⁷Co, ⁶⁰Cu, ⁶¹Cu, ⁶²Cu, ⁶⁴Cu, ⁶⁷Cu, ⁶⁷Ga, ⁶⁸Ga, ⁷²As ⁷⁶Br, ⁷⁷Br, ^{81m}Kr, ⁸²Rb, ⁸⁵Sr, ⁸⁹Sr, ⁸⁶Y, ⁹⁰Y, ⁹⁵Nb, ^{94m}Tc, ^{99m}Tc, ⁹⁷Ru, ¹⁰³Ru, ¹⁰⁵Rh, ¹⁰⁹Cd, ¹¹¹In, ¹¹³Sn, ^{113m}In, ¹¹⁴In, I¹²⁵, I¹³¹, ¹⁴⁰La, ¹⁴¹Ce, ¹⁴⁹Pm, ¹⁵³Gd, ¹⁵⁷Gd, ¹⁵³Sm, ¹⁶¹Tb, ¹⁶⁶Dy, ¹⁶⁶Ho, ¹⁶⁹Er, ¹⁶⁹Y, ¹⁷⁵Yb, ¹⁷⁷Lu, ¹⁸⁶Re, ¹⁸⁸Re, ²⁰¹Tl, ²⁰³Pb, ²¹¹At, ²¹²Bi or ²²⁵Ac.

Additional non-limiting exemplary detectable labels include a metal or a metal oxide. In particular embodiments, a metal or metal oxide is one or more of: gold, silver, copper, boron, manganese, gadolinium, iron, chromium, barium, europium, erbium, praseodynium, indium, or technetium. In additional embodiments, a metal oxide includes one or more of: Gd(III), Mn(II), Mn(III), Cr(II), Cr(III), Cu(II), Ffe(III), Pr(III), Nd(III) Sm(III), Tb(III), Yb(III) Dy(III), Ho(III), Eu(II), Eu(III), or Er(III).

Further non-limiting exemplary detectable labels include contrast agents (e.g., gadolinium; manganese; barium sulfate; an iodinated or noniodinated agent; an ionic agent or nonionic agent); magnetic and paramagnetic agents (e.g., iron-oxide chelate); nanoparticles; an enzyme (horseradish peroxidase, alkaline phosphatase, β-galactosidase, or acetylcholinesterase); a prosthetic group (e.g., streptavidin/biotin and avidin/biotin); a fluorescent material (e.g., umbelliferone, fluorescein, fluorescein isothiocyanate, rhodamine, dichlorotriazinylamine fluorescein, dansyl chloride or phycoerythrin); a luminescent material (e.g., luminol); or a bioluminescent material (e.g., luciferase, luciferin, aequorin).

Additional non-limiting examples of tags and/or detectable labels include enzymes (horseradish peroxidase, urease, catalase, alkaline phosphatase, beta-galactosidase, chloramphenicol transferase); enzyme substrates; ligands (e.g., biotin); receptors (avidin); GST-, T7-, His-, myc-, HA- and FLAG®-tags; electron-dense reagents; energy transfer molecules; paramagnetic labels; fluorophores (fluorescein, fluorscamine, rhodamine, phycoerthrin, phycocyanin, allophycocyanin); chromophores; chemi-luminescent (imidazole, luciferase, acridinium, oxalate); and bio-luminescent agents.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, colon cancer, glioma, cervical cancer, hepatocellular, thyroid cancer, or stomach cancer.

As used herein, a “metastatic cancer” is a cancer that spreads from where it originated to another part of the body.

As used herein, a “cancer cell” are cells that have uncontrolled cell division and form solid tumors or enter the blood stream.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

As used herein, an active ingredient (a.i.) can be an anticancer agent. An anticancer agent refers to any drug or compound used for anticancer treatment. These include any drug that renders or maintains a clinical symptom or diagnostic marker of tumors and cancer, alone or in combination with other compounds that reduces or maintains a state of remission, reduction, remission, prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin Kl-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives (as defined for imaging agents) thereof.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Immune response” broadly refers to the antigen-specific responses of lymphocytes to foreign substances. The terms “immunogen” and “immunogenic” refer to molecules with the capacity to elicit an immune response. All immunogens are antigens, however, not all antigens are immunogenic. An immune response disclosed herein can be humoral (via antibody activity) or cell-mediated (via T cell activation). The response may occur in vivo or in vitro. The skilled artisan will understand that a variety of macromolecules, including proteins, nucleic acids, fatty acids, lipids, lipopolysaccharides and polysaccharides have the potential to be immunogenic. The skilled artisan will further understand that nucleic acids encoding a molecule capable of eliciting an immune response necessarily encode an immunogen. The artisan will further understand that immunogens are not limited to full-length molecules, but may include partial molecules.

As used herein, “viral load”, also known as “viral burden,” “viral titer”, “viral level” or “viral expression” in some embodiments, is a measure of the severity of a viral infection, and can be calculated by estimating the amount of virus in an infected organism, an involved body fluid, or a biological sample.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

The term “micelle” as used herein refers to aggregates of amphiphilic block copolymers in selective solvents. These block copolymers generally have molecular weight one or two orders of magnitude larger than traditional surfactant molecules. This size discrepancy means that the exact chemical formula of a given copolymer can give rise to very different properties such as solubility or aggregation conditions.

The term “unimer” as used herein refers to a subunit of a dynamic micelle. Dynamic micelles from aggregates under some conditions and break into unimers under other conditions. Unimers conceptually are the micelle-equivalent of the monomers that make up polymers.

The term “foam cell” as used herein refers to fat-laden cells with a M2 macrophage-like phenotype. They contain low density lipoproteins (LDL) Some foam cells are derived from smooth muscle cells and present a limited macrophage-like phenotype. Foam cells can become a problem when they accumulate at particular foci thus creating a necrotic center of atherosclerosis.

### Modes for Carrying Out the Disclosure

### Formulation Methods

In one aspect, provided is a formulation process directed to incubation of plant virus nanoparticles (VNPs) with nonionic triblock-copolymers with the purpose of facilitating the bioconjugation of hydrophobic ligands. Exemplary nonionic triblock-copolymers include, but are not limited to, surfactants such as Pluronic F127 (F127) and equivalents thereof.

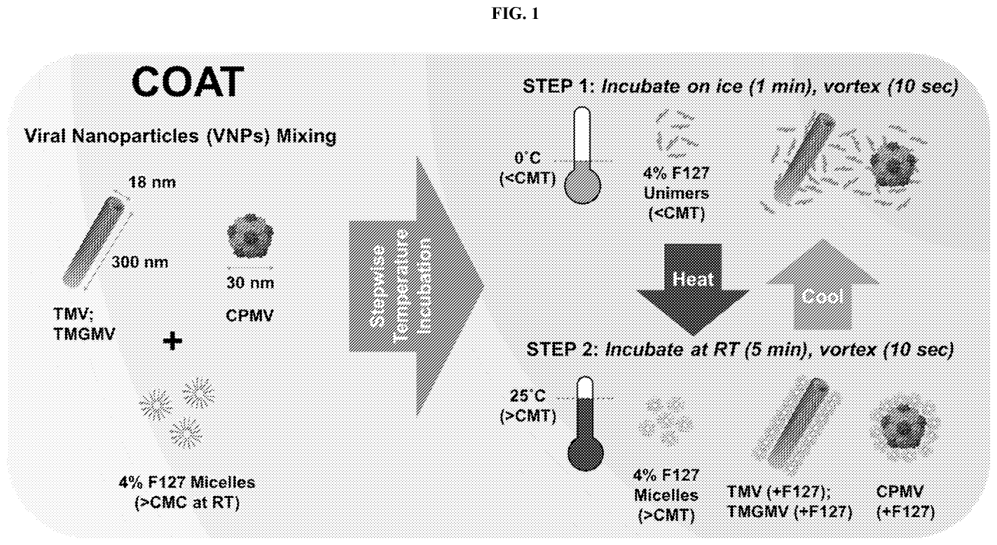

In some embodiments, this process may comprise the following steps: a) admixing a viral nanoparticle (VNP) with an effective amount of about 4 wt % of a poloxamer such as Pluronic F127 (F127) or an equivalent thereof to prepare a first mixture at a temperature of about 25° C.; b) cooling the mixture conjugate at a temperature below about 19.5° C. to promote the poloxamer such as F127 unimerization and then returning the mixture to a temperature of about 25° C. to promote the poloxamer such as F127 micellization; and c) mixing the mixture prepared by step b). In further embodiments, this process may further comprise the repetition of steps b) and c) at least twice or the repetition of steps b) and c) at least thrice. In some embodiments, any one or more of the following may be true: the effective amount of poloxamer in step a) is from 3.6 wt % to 4.4% wt %, the step a) temperature is from 23.75° C. to 26.25° C., the step b) unimerization temperature is from 18.5° C. to 20.5° C., and/or the step b) micellization temperature is from 23.75° C. to 26.25° C. The temperatures and wt % are illustrative only and they facilitate the phase changes of the composition. For example as illustrated in FIG. 1, the samples are first incubated on ice to promote the formation of F127 unimers before shifting to room temperature (RT) to promote F127 micellization and subsequent plant VNP coating.

In some embodiments, about 15 to about 45 molar equivalents of the poloxamer such as F127 or an equivalent per coat protein (CP) of the VNP or about 28 molar equivalents of the poloxamer such as F127 or an equivalent per CP is be used in step a) of the process. In some embodiments, the poloxamer molar equivalent is from 13.5 to 50 molar equivalents or an equivalent per coat protein (CP) of the VNP or from 25 to 31 molar equivalents of the poloxamer or an equivalent per CP is be used in step a) of the process.

Non-limiting examples of the VNP used in the process is selected from: tobacco mosaic virus (TMV), cowpea mosaic virus (CPMV), tobacco mild green mosaic virus (TMGMV), or a modified equivalent thereof. In further embodiments, the modified equivalent VNP can be selected from: TMV-lysine, TMV-lysine with a Lys substitution at amino acid position 158, CPMV-lysine, CPMV- with a Lys substitution at amino acid position 158, chemically conjugated TMV, chemically conjugated CPMV, chemically conjugated TMGMV, TMV-Cys5.5, ligand-modified TMV, ligand-modified CPMV, ligand-modified TMGMV, Avermectin-TMGMV or TMGMV-Cys5

In further embodiments, the ligand-modified TMV, ligand-modified CPMV, or ligand-modified TMGMV comprises a ligand selected from: a peptide epitope for a vaccine, a peptide epitope for a COVID vaccine, a targeting peptide, an agrochemical, a detectable label, a small molecule, a fluorophore, or a peptide selected from CH401-Rat (SEQ ID NO: 1), Myostatin 1 (SEQ ID NO: 2), COVID-106 (SEQ ID NO: 3), COVID-153 (SEQ ID NO: 4), COVID-454 (SEQ ID NO: 5), COVID-826 (SEQ ID NO: 6), F3 (SEQ ID NO: 7), ScG3 (SEQ ID NO: 8), or ApoAI-4FN (SEQ ID NO: 9) In still further embodiments, the ligand selected from the list is hydrophobic

In another embodiment, the process can comprise the following steps: a) admixing a viral nanoparticle (VNP) with an effective amount of about 4 wt % of a poloxamer such as Pluronic F127 (F127) or an equivalent thereof and also a hydrophobic Cy5.5-NHS ligand at about 2 molar equivalents per CP to prepare a first mixture at a temperature of about 25° C.; b) cooling the mixture conjugate at a temperature below about 19.5° C. to promote the poloxamer such as F127 unimerization and then returning the mixture to a temperature of about 25° C. to promote the poloxamer such as F127 micellization; and c) mixing the mixture prepared by step b). In a further aspect, this process may further comprise the repetition of steps b) and c) at least twice or the repetition of steps b) and c) at least thrice. In other embodiments, steps b) and c) can be omitted. In other embodiments, the VNP and the poloxamer are mixed with a Cy5.5-NHS ligand at 1.5 to 2.5 molar equivalents per CP in step a). In further embodiments, the VNP used in this Cy5.5-NHS-focused process comprises TMV. The temperatures and wt % are illustrative only and they facilitate the phase changes of the composition.

In another embodiment, the process can comprise the following steps: a) admixing a viral nanoparticle (VNP) with an effective amount of about 4 wt % of a poloxamer such as Pluronic F127 (F127) or an equivalent thereof and also with an Avermectin ligand at about 5 molar equivalents per CP, in the presence of Cu(I)-catalyzed azide-alkyne cycloaddition to prepare a first mixture at a temperature of about 25° C.; b) cooling the mixture conjugate at a temperature below about 19.5° C. to promote the poloxamer such as F127 unimerization and then returning the mixture to a temperature of about 25° C. to promote the poloxamer such as F127 micellization; and c) mixing the mixture prepared by step b). In a further aspect, this process may further comprise the repetition of steps b) and c) at least twice or the repetition of steps b) and c) at least thrice. In other embodiments, steps b) and c) can be omitted. In other embodiments, the VNP and poloxamer are mixed with avermectin ligand at 4.5 to 5.5 molar equivalents per CP in step a). The temperatures and wt % are illustrative only and they facilitate the phase changes of the composition.

In some embodiments, the VNP used in step a) of the present process can comprise a maleimide-bearing VNP-M(PEG)₄. In further embodiments, the VNP can comprise maleimide-bearing M(PEG)₄TMV or maleimide-bearing M(PEG)₄CPMV.

In another embodiment, the process can comprise the following steps: a) admixing a viral nanoparticle (VNP) comprising a maleimide-bearing CPMV-M(PEG)₄ with an effective amount of about 4 wt % of a poloxamer such as Pluronic F127 (F127) or an equivalent thereof and a peptide to prepare a first mixture at a temperature of about 25° C.; b) cooling the mixture conjugate at a temperature below about 19.5° C. to promote the poloxamer such as F127 unimerization and then returning the mixture to a temperature of about 25° C. to promote the poloxamer such as F127 micellization; and c) mixing the mixture prepared by step b). In a further aspect, this process may further comprise the repetition of steps b) and c) at least twice or the repetition of steps b) and c) at least thrice. In some embodiments, any one or more of the following may be true: the step a) wt % of the poloxamer is 9% to 11%, the effective amount of poloxamer in step b) is from 3.6 wt % to 4.4% wt %, the step b) temperature is from 23.75° C. to 26.25° C., the step c) unimerization temperature is from 18.5° C. to 20.5° C., and/or the step c) micellization temperature is from 23.75° C. to 26.25° C. The temperatures and wt % are illustrative only and they facilitate the phase changes of the composition.

In a further embodiment, the peptide used in step a) of the process peptide is selected from a COVID19 epitope, a HER2 epitope, cyclosporine A peptide, MyoI, or a peptide selected from CH401-Rat (SEQ ID NO: 1), Myostatin 1 (SEQ ID NO: 2), COVID-106 (SEQ ID NO: 3), COVID-153 (SEQ ID NO: 4), COVID-454 (SEQ ID NO: 5), COVID-826 (SEQ ID NO: 6), F3 (SEQ ID NO: 7), ScG3 (SEQ ID NO: 8), or ApoAI-4FN (SEQ ID NO: 9). In further embodiments, the peptide is hydrophobic. In some embodiments, the peptide comprises a Cys-terminal or N-terminal GGSC or GGGC linker. In further embodiments, the GGSC or GGGC linker on the peptide reacts with a heterobifunctional maleimide linker SM(PEG)₄

In another aspect, provided is a formulation process directed towards the incubation of peptides with nonionic triblock-copolymers such as F127 with the purpose of facilitating the bioconjugation of said peptides.

This process can comprise the following steps: a) dialyzing a water-insoluble peptide dissolved in DMSO and about 10 wt % of a poloxamer such as F127 or an equivalent thereof to form a peptide-loaded micelle (a FORM peptides); b) mixing the FORM peptide with an M(PEG)₄-VNP with an effective amount of about 4 wt % of a poloxamer such as Pluronic F127 (F127) or an equivalent thereof to prepare a first mixture at a temperature of about 25° C.; c) cooling the mixture conjugate at a temperature below about 19.5° C. to promote the poloxamer such as F127 unimerization and then returning the mixture to a temperature of about 25° C. to promote the poloxamer such as F127 micellization; and d) mixing the mixture prepared by step c). In further embodiments, this process may further comprise the repetition of steps b) and c) at least twice or the repetition of steps b) and c) at least thrice. The temperature and wt % are illustrative only and are selected to facilitate phase change of the composition.

In some embodiments, about 15 to about 45 molar equivalents of F127 or an equivalent per coat protein (CP) of the VNP or about 28 molar equivalents of the poloxamer such as F127 or an equivalent per CP can be used in step a) of the process. In some embodiments, the poloxamer molar equivalent is from 13.5 to 50 molar equivalents or an equivalent per coat protein (CP) of the VNP or from 25 to 31 molar equivalents of the poloxamer or an equivalent per CP is be used in step a) of the process.

### Compositions

Further provided is a particle prepared by a process as described herein. The particles are useful diagnostically and therapeutically. In some embodiments, the particle comprises a VNP chemically conjugated to a plurality of small molecules. Exemplary particles are illustrative and not intended as limiting.

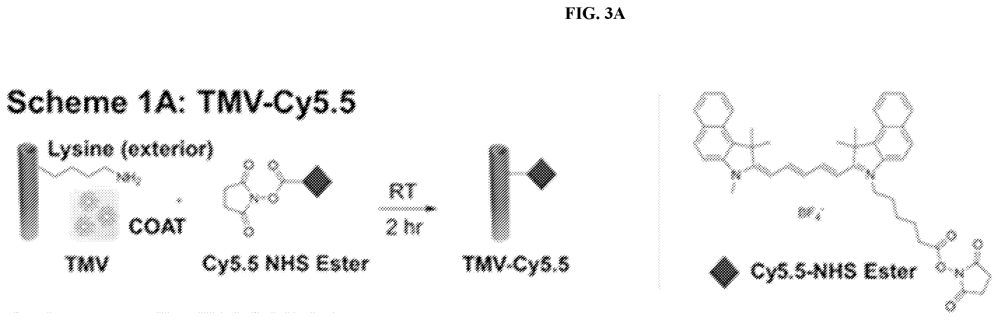

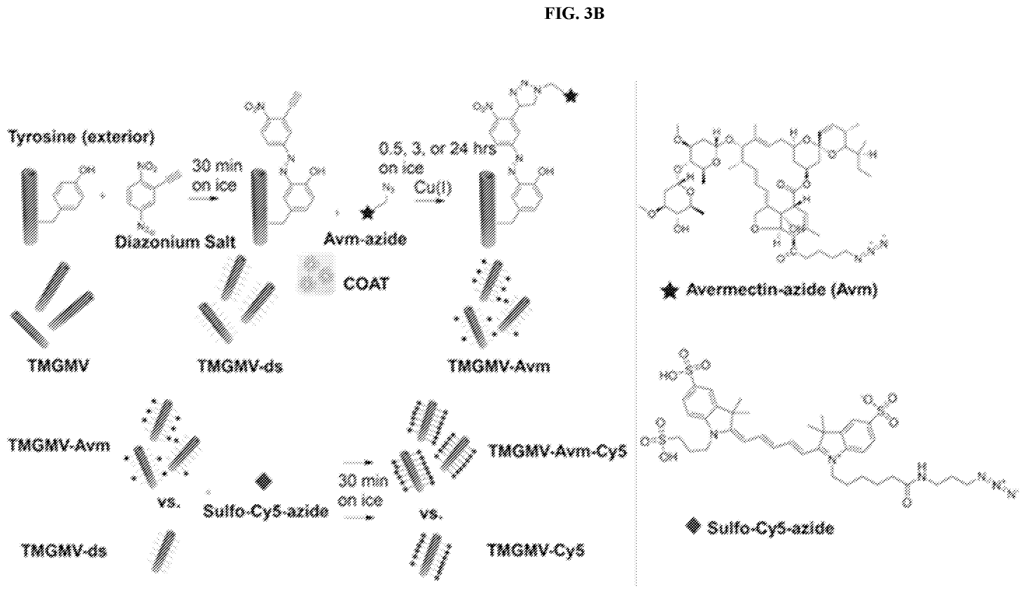

One such exemplary particle comprises a TMV particle that further comprises a plurality of lysine residues chemically conjugated to an average of 110 Cy5.5 dye molecules through lysine-NHS ester linkages and is illustrated in FIG. 3A. Another exemplary particle comprises a TMGMV particle that further comprises a plurality of tyrosine residues chemically conjugated to diazonium salts. The modified TMGVM particle further comprises an average of 230 avermectin-azide molecules conjugated to the modified tyrosine residues by Cu(I)-catalyzed azide-alkyne cycloaddition (click chemistry) as shown in FIG. 3B.

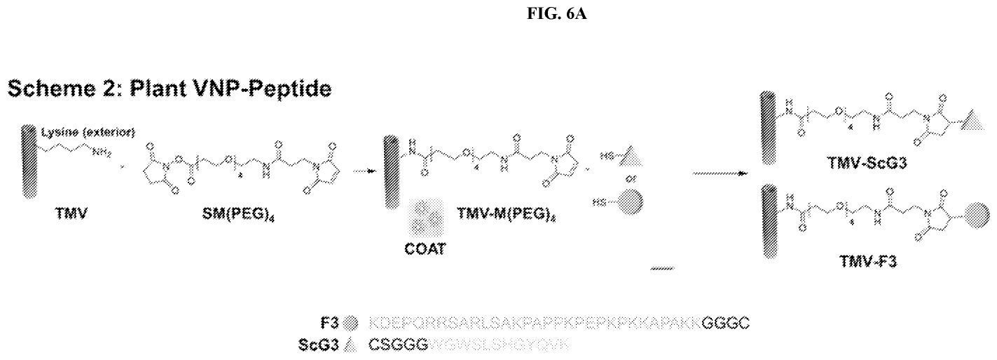

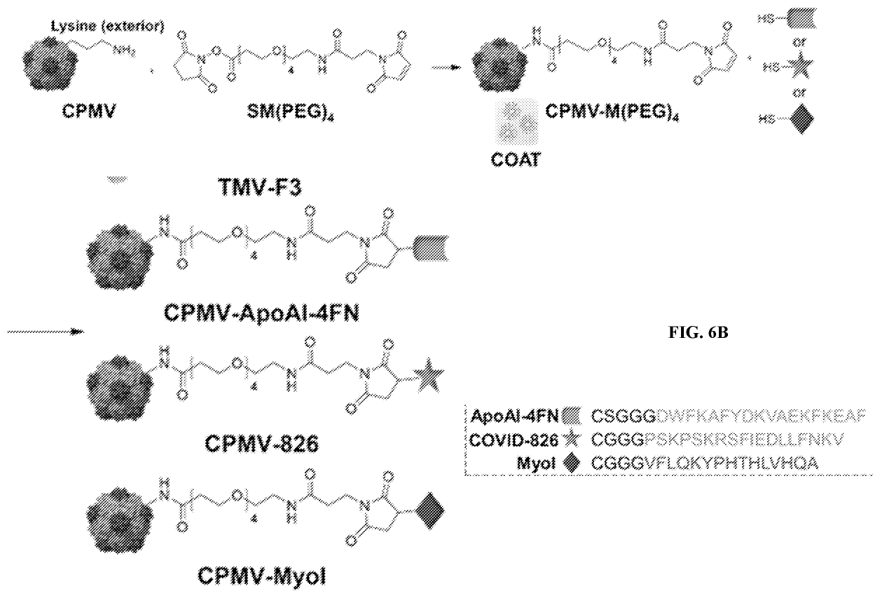

Another exemplary particle comprises a TMV particle that further comprises a plurality of lysine residues chemically conjugated to SM(PEG)₄ molecules. The modified TMV particle further comprises F3 (SEQ ID NO: 7) or ScG3 (SEQ ID NO: 8) peptides conjugated to the PEGylated TMV particle through maleimide thiol chemistry as shown in FIG. 6A. Another exemplary particle comprises a CMPV particle that further comprises a plurality of lysine residues chemically conjugated to SM(PEG)₄ molecules. The modified CMPV particle further comprises a plurality of peptides conjugated to the PEGylated CMPV particle through maleimide thiol chemistry chosen from the list: CH401-Rat (SEQ ID NO: 1), Myostatin 1 (SEQ ID NO: 2), COVID-106 (SEQ ID NO: 3), COVID-153 (SEQ ID NO: 4), COVID-454 (SEQ ID NO: 5), COVID-826 (SEQ ID NO: 6), or ApoAI-4FN (SEQ ID NO: 9) as shown in FIG. 6B.

In another aspect, provided is a composition comprising the resulting product of any process disclosed herein and a carrier. In some embodiments, the carrier is a pharmaceutically acceptable carrier. The composition can contain a further active agent selected for the particular use, e.g., anti-cancer or an anti-viral agent.

In another aspect, provided herein is a composition comprising, consisting essentially of, or consisting of the combination of formulations comprising a composition as provided herein, and at least one carrier, such as a pharmaceutically acceptable carrier or excipient. In one aspect, the composition further comprises a preservative or stabilizer.

In one embodiment, this technology relates to a composition comprising a combination of compositions or formulations as described herein and a carrier.

In another embodiment, this disclosure provides a pharmaceutical composition comprising an effective amount or a therapeutically effective amount of a composition formulations as described herein and a pharmaceutically acceptable carrier.

Compositions, including pharmaceutical compositions comprising, consisting essentially of, or consisting of the composition alone or in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, oral, buccal, rectal, sublingual, or transdermal administration routes. In some cases, parenteral administration comprise, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL® PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOLR), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, polysorbate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the compositions are administered to a subject suffering from a condition as disclosed herein, such as a human, either alone or as part of a pharmaceutically acceptable formulation, once a week, once a day, twice a day, three times a day, or four times a day, or even more frequently.

Administration of the composition alone or in combination with an additional therapeutic agent and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprise, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

In some embodiments, the sample may be an upper respiratory specimen, such as a nasopharyngeal (NP) specimen, an oropharyngeal (OP) specimen, a nasal mid-turbinate swab, an anterior nares (nasal swab) specimen, or nasopharyngeal wash/aspirate or nasal wash/aspirate (NW) specimen.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis. In one aspect, a sample is obtained to determine the presence or absence of cancer or a tumor, or a viral infection, e.g., COVID.

### Therapeutic Methods

In another aspect, provided is a delivery method for a composition that comprises administering to a subject in need thereof a formulation comprising the resulting product of any process disclosed herein or a combination of such a formulation and a carrier. In some embodiments, the subject is a mammal. In further embodiments, the subject is a human patient.

In another aspect, provided is a method of promoting cholesterol efflux from a foam cell or inhibiting the growth of a cancer cell comprising contacting the cell with an effective amount of a composition disclosed herein. In some embodiments, the composition comprises ligand-modified TMV, ligand-modified CPMV, or ligand-modified TMGMV that is modified with the ligand ApoAI-4FN (SEQ ID NO: 9).

In some embodiments, administering is selected from intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, or inhalation. In some embodiments, administering is intravenous.

### Coronavirus Vaccines

In a further aspect, the composition comprises an antigenic peptide from a coronavirus, e.g, as disclosed herein, that is administered to a subject in need thereof, to induce an immune response in the subject in need thereof, e.g., an immune response against a coronavirus, e.g., a COVID-2 infection. In one aspect, the antigenic peptide is a fragment of the SARs S protein. The compositions can further comprise an adjuvant and the method can be further combined with other appropriate therapies. The composition may be atomized by a nebulizer inhalation system prior to or during administration. The nebulizer system may be a portable nebulizer for whole respiratory tract drug delivery. The composition may be administered by subcutaneous injection, intramuscular injection, or intraperitoneal injection (i.p).

Also provided is a method for one or more of: inhibiting or preventing a symptomatic COVID infection, treating a COVID infection, immunizing against a COVID infection, comprising, or consisting essentially of, or yet further consisting of administering to a subject in need thereof an effective amount of a composition as disclosed herein further comprising an antigenic peptide from a coronavirus, to the subject. The compositions can further comprise an adjuvant and the method can be further combined with other appropriate therapies. The composition may be atomized by a nebulizer inhalation system prior to or during administration. The nebulizer system may be a portable nebulizer for whole respiratory tract drug delivery. The composition may be administered by subcutaneous injection, intramuscular injection, or intraperitoneal injection (i.p).

In some embodiments, the subject is a mammal. In further embodiments, the subject is a human patient.

### Cancer Therapy

The compositions having active agents directed to inhibiting the growth of a cancer cell, are useful in methods are useful for cell labeling, diagnostics and therapies. The contacting of the cell in vitro or in vivo. The cell can be from a mammal, e.g., a human cell. When contacted in vivo, the method is useful to test the effectiveness of the therapy against a cancer cell. The cancer can be obtained from a biopsy or an established cell line. These are commercially available from vendors, such as the American Type Culture Collection (ATCC). When practiced in vivo, the method is useful as an animal model to test combination therapies or to test the therapy against cancer types or in the treatment of human disease.

In another aspect, the composition can further comprise another anticancer agent and contacted with the cell.

One of skill in the art can determine when the composition has been delivered by methods know to the skilled artisan and briefly described herein, e.g., by determining if the cell or target has been modified by the composition, e.g., the growth of a cancer cell has been inhibited by the delivery of an anti-cancer drug or therapy.

Further disclosed herein are methods for inducing an immune response in a subject consisting essentially of, or yet further consisting administering the composition as disclosed herein.

Further disclosed herein are methods for treating cancer in a subject in need thereof, comprising, or alternatively consisting essentially of, or yet further consisting of administering to the subject the composition as disclosed herein.

In some embodiments, a subject is a mammal. In some embodiments, a subject is a human. In some embodiments, a subject has a condition. In some embodiments, a subject has cancer. In some embodiments, a cancer is selected from melanoma, breast cancer, prostate cancer, lung cancer, ovarian cancer, skin cancer, bladder cancer, pancreatic cancer, gastric cancer, esophageal cancer, colon cancer, glioma, cervical cancer, hepatocellular cancer, or thyroid cancer. In some embodiments, the cancer is primary or metastatic cancer. In some embodiments, the cancer is metastatic or primary ovarian cancer or breast cancer. In some embodiments, the cancer has metastasized.

In some embodiments, administering is selected from intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, or inhalation. In some embodiments, administering is intravenous.

The methods and compositions disclosed herein may further comprise or alternatively consist essentially of, or yet further consists of administering to the subject an anti-tumor therapy other than the composition. In some embodiments, anti-tumor therapy may include different cancer therapy or tumor resection. The additional therapeutic can be combined in the same composition or separately administered.

In some embodiments, the composition is provided to prevent the symptoms of cancer from occurring in a subject that is predisposed or does not yet display symptoms of the cancer.

In some embodiments, the composition disclosed herein may be delivered or administered into a cavity formed by the resection of tumor tissue (i.e. intracavity delivery) or directly into a tumor prior to resection (i.e. intratumoral delivery). In some embodiments. In some embodiments, the administering is intravenous.

In some embodiments, any of the composition disclosed herein are administered to the subject at least 1, 2, 3, 4, 5, 6, 7, 8, 9, or 10 times a day. In some embodiments, the composition is administered to the subject at least 1, 2, 3, 4, 5, 6, 7, 8, 9, 10, 11, 12, 13, 14, 15, 16, 17, 18, 19, or 20 times a week. In some embodiments, any of the composition is administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the composition disclosed herein is administered to the subject at least every 1, 2, 3, 4, 5, 6, 7, 8, 9, or 10 days.

In some embodiments, the method and compositions provided herein, comprise, or alternatively consist essentially of, or yet further consist of inhibiting metastatic potential of the cancer, reduction in tumor size, a reduction in tumor burden, longer progression free survival, or longer overall survival of the subject.

In some cases, the composition with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, chemotherapy. In other cases, the first-line treatment comprise, or consists essentially of, or yet further consists of, radiation therapy. A skilled artisan would readily understand that different first-line treatments may be applicable to different type of cancers.

In some cases, the composition is administered as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In one aspect, the methods or compositions further comprise administration of an additional therapeutic agent. In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

Additional therapeutic agents include for example an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULAR, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. In some cases, the additional therapeutic agent comprise, or consists essentially of, or yet further consists of, pembrolizumab, nivolumab, tremelimumab, or ipilimumab.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the conjugate or composition is administered in combination with a radiation therapy.

### Kits

In another aspect, provided is a kit that comprises the composition that comprises resulting product of any process disclosed herein and instructions for use. In some embodiments, the kit comprises a formulation comprising the resulting product of any process disclosed herein and a carrier. In further embodiments, the carrier is a pharmaceutically acceptable carrier.

In one particular aspect, the present disclosure provides kits for performing the methods of this disclosure as well as instructions for carrying out the methods of the present disclosure. The kit comprises, or alternatively consists essentially of, or yet further consists of one or more of the conjugate or composition and instructions for use. In a further aspect, the instruction for use provide directions to conduct any of the methods disclosed herein.

The kits are useful for detecting the presence of disease such as breast cancer or the presence of a coronavirus in a biological sample e.g., any bodily fluid including, but not limited to, e.g., sputum, serum, plasma, lymph, cystic fluid, urine, stool, cerebrospinal fluid, acitic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### Experimental

Bioconjugation involves the formation of covalent bonds between large biomolecules and smaller synthetic structures. One of the main applications of bioconjugation is the conversion of such biomolecules into nanocarriers that encapsulate or display active ingredients for medical or agricultural applications. Some nanocarriers are designed to encapsulate their cargo without bioconjugation, including liposomes with hydrophilic exterior and hydrophobic interior compartments. Others are based on biopolymers,¹ protein nanoparticles,² or viruses, and bioconjugation covalently attaches the cargo to the external or internal surface. The cargo may comprise drugs, therapeutic peptides/proteins, peptide/protein antigens, cell/organelle-specific peptide ligands for cell targeting and trafficking, fluorophores or contrast agents for tracking and imaging, as well as agrochemicals for pesticide delivery.³ Nanocarriers offer many advantages for cargo delivery including the enhanced solubility of hydrophobic ligands, controlled and sustained drug release, targeted delivery to specific tissues, and multivalent ligand display, which allows the number, spacing, and avidity of ligands to be optimized.⁴

Plant virus nanoparticles (plant VNPs) are among the most versatile of nanocarrier platforms. They are biocompatible and biodegradable but are non-infectious in mammals and thus offer a higher degree of safety than the animal viruses used as gene-delivery vectors. They have also evolved as mobile nanocarriers to navigate plants and soil and can therefore be repurposed for precision farming. Plant VNPs come in a variety of shapes and sizes, including icosahedral forms and high-aspect-ratio nanorods or filaments with sizes in the range 30-500 nm.⁵ Many plant VNPs are self-assembled supramolecular complexes based on simple coat protein (CP) units featuring solvent-accessible interior and exterior surfaces whose various amino acids provide ‘chemical handles’ for bioconjugation. Applicant have previously used tobacco mosaic virus (TMV-Lys, with a Lys substitution at amino acid position 158),⁶ tobacco mild green mosaic virus (TMGMV),⁷ and cowpea mosaic virus (CPMV)^{8,9} for various applications targeting human and plant health. Each plant VNP has been extensively characterized and the surface chemistry is understood,^{7,10,11} allowing functionalization for diverse applications.¹²⁻¹⁴ Furthermore, multifunctional conjugation can be achieved by the multiplexed targeting of lysine, cysteine, glutamate/aspartate and/or tyrosine side chains.¹⁵

Although plant VNP scaffolds offer a high degree of tunability, the conjugation of hydrophobic cargo molecules can reduce overall solubility, affecting structural integrity and yields. Applicant therefore evaluated the use of Pluronic F127 (F127), a nonionic triblock-copolymer comprising a core block of repeating hydrophobic poly(propylene oxide) units capped by a terminal block of repeating poly(ethylene oxide) units at each end of the molecule. This surfactant is approved as a pharmaceutical excipient and has been used as a wetting/solubilizing agent in the manufacture of micro/nanoparticles and the micellization of hydrophobic drugs or peptides.¹⁶⁻²³ It has also been used as a coating to protect TMV and ferritin.²⁴ However, to the best of our knowledge, it has not been used thus far to facilitate the bioconjugation of plant VNPs to hydrophobic ligands.

F127 does not covalently modify nanoparticles or their payloads, but chemically-modified derivatives of F127 have been fused to ligands and drugs either by coating nanoparticles composed of PLGA, SPION, or chitosan, or precipitation within them.²⁵⁻²⁷ Applicant exploited the intrinsic ability of unmodified F127 to coat plant VNPs and solubilize hydrophobic ligands to determine whether a preincubation step can facilitate bioconjugation yielding stable and soluble compositions. Applicant compared plant VNP-polymer precoat (COAT) and ligand formulation plus plant VNP-polymer precoat (FORMCOAT) strategies using a ligand library featuring near-infrared dyes for biomedical imaging, pesticides for precision farming, therapeutic proteins for drug delivery, disease-targeting peptides, and peptide antigen vaccine candidates (Table 1). Applicant tested N-hydroxysuccinimide (NHS) ester-amine coupling, copper(I)-catalyzed azide-alkyne cycloaddition (click chemistry), and maleimide-thiol coupling in the presence and absence of F127, then characterized the products by digital imaging, sodium dodecylsulfate polyacrylamide gel electrophoresis (SDS-PAGE), dynamic light scattering (DLS), transmission electron microscopy (TEM), and ultraviolet-visible (UV-Vis) spectroscopy. Applicant focused on plant VNP-ligand conjugates that undergo visible aggregation when prepared using conventional protocols.

### Results and Discussion

### Ligand Selection

Applicant selected 11 candidates from a library of ligands that have previously shown a tendency to trigger the aggregation of conjugated plant VNPs (Table 1). Applicant included cyanine5.5 (Cy5.5) because it is widely used for biomedical imaging. 28.29 The sulfonated version (log P=1.91) is highly soluble in water and allows stable conjugation, whereas the non-sulfonated version (log P=4.94) has proven more challenging in our hands. Applicant obtained the NHS ester of non-sulfonated Cy5.5 for conjugation to solvent-exposed lysine side chains on CPMV and TMV.

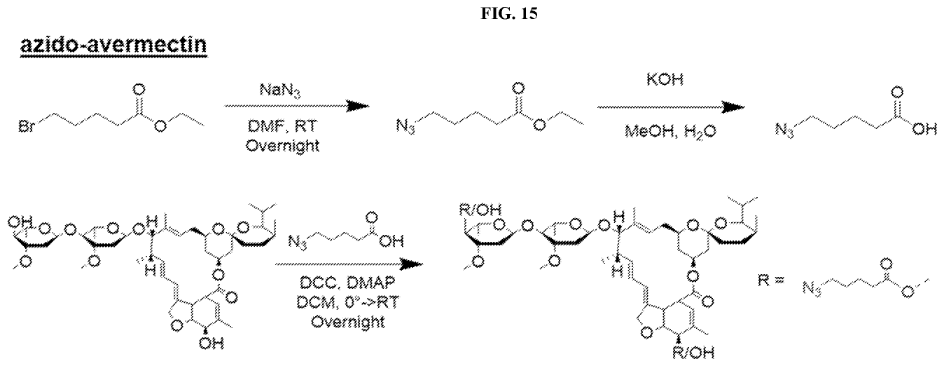

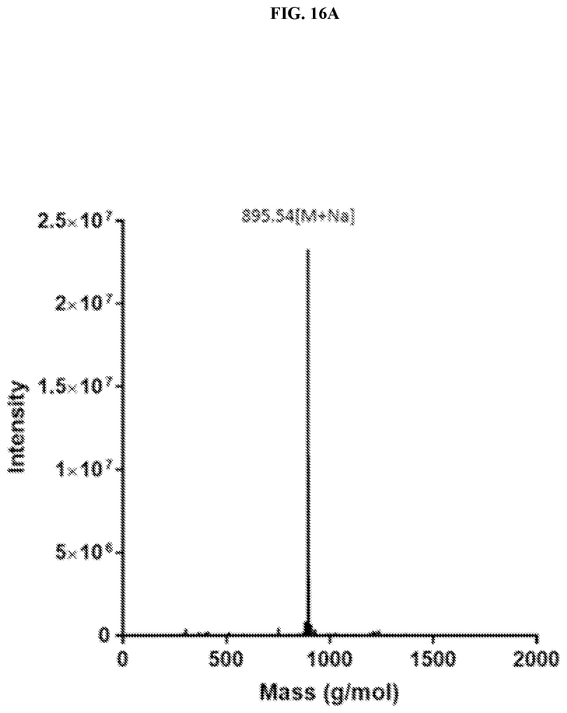

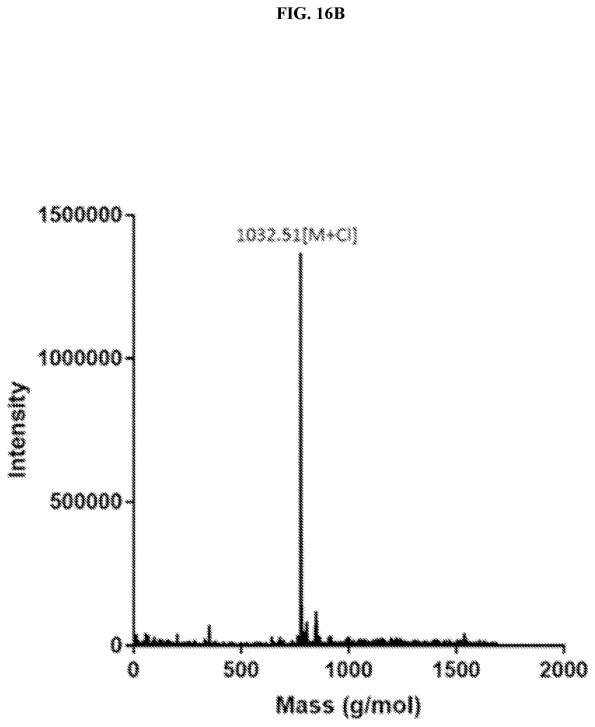

Applicant included Avermectin as a model hydrophobic small-molecule drug/pesticide (log P=4.37) representing other hydrophobic insecticides such as moxidectin (log P=5.30) and afidopyropen (log P=3.08) as well as drugs such as doxorubicin (log P=1.41) and paclitaxel (log P=3.20). Nanocarrier conjugates are promising formulations to stabilize avermectin and enhance its soil mobility.^{7,30} Avermectin azide (Avm) was synthesized in-house (FIG. 15) and was characterized by electrospray ionization mass spectrometry (ESI-MS) (FIGS. 16A-16B).

Finally, Applicant included nine peptides with medical applications. CH401R (SEQ ID NO: 1) is an epitope of human epidermal growth factor receptor 2 (HER2) and a candidate cancer vaccine. Applicant previously demonstrated the ability of CPMV-CH401R conjugates to delay tumor progression in mouse models of HER2⁺ breast cancer.³¹ MyoI (SEQ ID NO: 2) is a peptide epitope of myostatin, which has been proposed as a muscle growth vaccine.^{32,33} Four peptide epitopes derived from the SARS-COV-2 S-protein (SEQ ID NOs: 3-6) were selected as candidates for a COVID-19 vaccine.³⁴⁻³⁸ F3 (SEQ ID NO: 7) and ScG3 (SEQ ID NO: 8) were selected as model cell-targeting peptides. F3 targets the cell shuttle molecule nucleolin, and Applicant has shown that drug-loaded plant VNPs displaying this peptide are more efficient at killing cancer cells.³⁹ ScG3 targets an inflammatory regulator (S100 calcium-binding protein A9), and Applicant previously used plant VNPs displaying this peptide for the imaging of atherosclerotic plaques.^{39,40} Finally, 4FN (SEQ ID NO: 9) is a therapeutic peptide derived from apolipoprotein A1 that promotes cholesterol efflux from foam cells and also suppresses tumor activity.⁴¹ All nine peptides were synthesized with a GGGSC or GGGC extension to enable conjugation via the heterobifunctional maleimide linker SM(PEG)₄. This simultaneously reacts with terminal cysteine residues on the peptides and a succinimidyl group that binds solvent-exposed lysine residues on CPMV and TMV. The molecular weight of the peptides ranged from 1741 to 3707 Da, with log P values of −0.89 to 1.53 (determined using A Log PS), isoelectric points of 6.21 to 11.08 (determined using PepCalc), and a net charge at pH 7 of −0.1 to 7.9 (also determined using PepCalc)

TABLE 1
Summary of ligand properties.

| | | | | Iso-electric | Net | Estimated | Observed |
|---|---|---|---|---|---|---|---|
| | Peptide/Small | MW | Log | point | charge | water | water |
| Ligand Type | Molecule* | (Da) | P# | (pH)\hat{ } | @ pH 7\hat{ } | solubility\hat{ } | solubility |
| Peptide | CH401-Rat | 2922 | 1.19 | 8.54 | 0.9 | Poor | Poor |
| epitope | Myostatin 1 | 2092 | 1.06 | 8.96 | 1.2 | Poor | Good |
| for vaccine | COVID-106 | 2043 | 0.84 | 9.12 | 0.9 | Poor | Poor |
| | COVID-153 | 2158 | 0.98 | 9.73 | 2 | Poor | Poor |
| | COVID-454 | 2164 | 0.32 | 8.79 | 0.9 | Poor | Poor |
| | COVID-826 | 2380 | 0.27 | 10.08 | 1.9 | Good | Good |
| Targeting | F3 | 3707 | 0.81 | 11.08 | 7.9 | Good | Good |
| peptide | ScG3 | 1809 | 0.27 | 8.96 | 1 | Poor | Good |
| Therapeutic | ApoAI-4FN | 2631 | 1.53 | 6.36 | −0.1 | Good | Good |
| peptide | | | | | | | |
| Agrochemical | Avermcetin- | 950 | 4.22 | | | | |
| | Azide* | | | | | | |
| Flurophore | Cy5.5 NHS | 768 | 3.08 | | | | |
| | Ester* | | | | | | |

#Calculated by ALOGPS2.1
\hat{ }Calculated by Pepcalc.org

| | Peptide/Small | |
|---|---|---|
| Ligand Type | Molecule* | Sequence |
| Peptide | CH401-Rat | YQDMVLWKDVFRKNNQLAPVGPSLC |
| epitope | Myostatin 1 | CGGGVFLQKYPHTHLVHQA |
| for vaccine | COVID-106 | CGGGFASTEKSNIIRGWIF |
| | COVID-153 | CGGGPFLGVYYHKNNKSWM |
| | COVID-454 | CGGGNNLDSKVGGNYNYLYR |
| | COVID-826 | CGGGPSKPSKRSFIEDLLFNKV |
| Targeting | F3 | KDEPQRRSARLSAKPAPPKPEPKPKKAPAKKGGGC |
| peptide | ScG3 | CSGGGWGWSLSHGYQVK |
| Therapeutic | ApoAI-4FN | CSGGGDWFKAFYDKVAEKFKEAF |
| peptide | | |
| Agrochemical | Avermectin- | |
| | Azide* | |
| Flurophore | Cy5.5 NHS | |
| | Ester* | |

#Calculated using ALOGPS2.1 (log P is the octanol/water partition coefficient, with higher
values representing increasing lipophilicity).
\hat{ }Calculated using PepCalc (the isoelectric point is the pH condition at which the net charge of
the peptide is zero).

Coating Plant VNPs with F127 (COAT Strategy)

### General Strategy

The plant VNPs were coated with F127 as a temporary scaffold in a procedure described as the COAT strategy (FIG. 1). F127 has a critical micelle concentration (CMC) of 0.725% (w/w) at 25° C., and Applicant used a 4% (w/w) solution for coating, which thus favors the formation of micelles.^{23,42} The plant VNP and F127 solutions were combined and the sample temperature was reduced to below the critical micelle temperature (CMT) of 19.5-21.5° C. to promote the homogeneous distribution of F127 unimers.⁴³ Then the temperature was raised to promote micellization and plant VNP coating. This process is also reversible (lowering the temperature again can promote ‘de-coating’ and plant VNP purification).⁴⁴

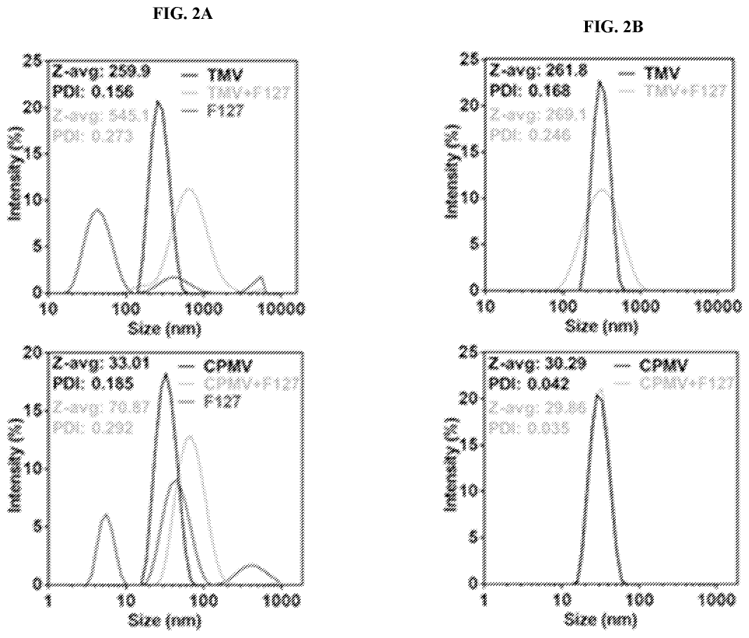

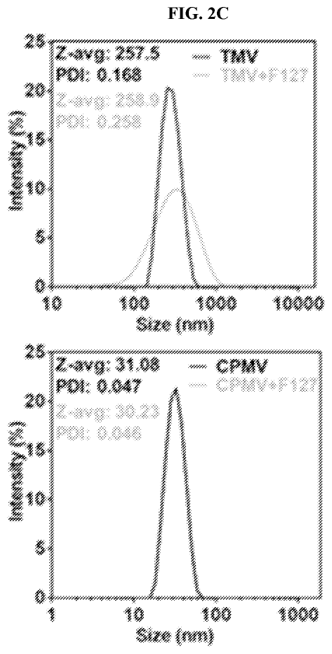

The COAT strategy was applied successfully to CPMV and TMV as confirmed by DLS (FIG. 2). The DLS intensity plots of plant VNP (+F127) particles showed an increase in diameter, which indicated successful coating with F127 micelles. The Z-average of TMV increased from 259.9 nm (untreated) to 545.1 nm (after the COAT process) and the polydispersity (PDI) increased from 0.165 to 0.273. Similarly, the Z-average of CPMV increased from 33.01 nm (untreated) to 70.87 nm (after the COAT process) and the PDI increased from 0.185 to 0.292. The plant VNP peaks after the COAT process were larger than the starting peaks of either plant VNP or F127. In the mixed sample, the disappearance of the characteristic F127 unimer, micellar, and supramolecular micellar peaks probably reflect favorable coating interactions on the plant VNP surface.

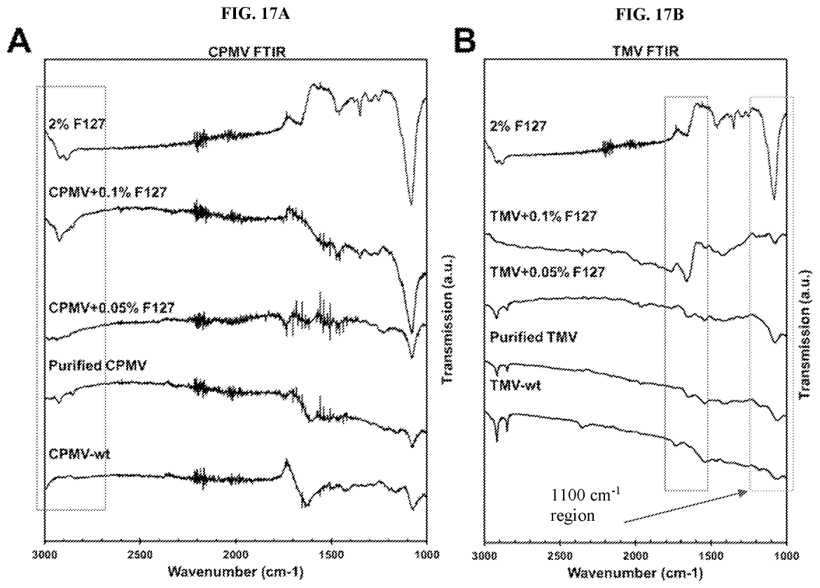

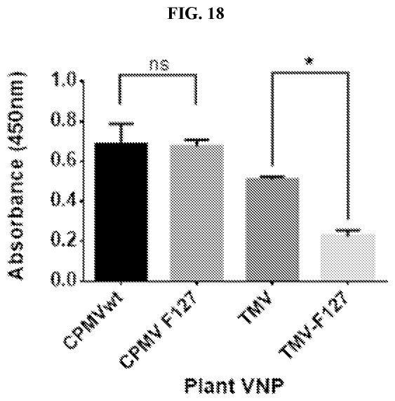

The DLS spectra of CPMV and TMV following ultracentrifugation at 4° C. or room temperature indicated that the F127 coating can be removed, although there was evidence of residual F127 still adsorbed to TMV after purification. (FIG. 2B, FIG. 2C). In contrast, no clear indications of residual F127 were present for purified CPMV (FIG. 2B, FIG. 2C). For CPMV, the Z-average and PDI after ultracentrifugation at 4° C. indicated the recovery of pure CPMV with a diameter of ˜30 nm and a narrow PDI (˜0.04). For TMV, the Z-average after ultracentrifugation at 4° C. matched that of untreated TMV (˜260 nm) but the PDI was still higher (0.26) than untreated TMV (0.17), which may indicate a population of TMV with residual surface polymer (FIG. 2C). Because room temperature is well above the CMT of 4% (w/w) F127, Applicant hypothesized that purification at room temperature would yield coated plant VNPs. This was not the case for CPMV, as no significant difference in Z-average or PDI was observed between purified CPMV and uncoated CPMV control (both ˜30 nm and PDI<0.05). (FIG. 2B). However, Z-average of the TMV particles was 269 nm (compared to 262 nm for untreated particles) and the PDI was 0.24 (compared to 0.17 for untreated particles), again suggesting presence of residual surface polymer. Applicant also detected residual F127 on TMV particles by Fourier transform infrared (FT-IR) spectroscopy (FIGS. 17A-B) and observed a weaker enzyme-linked immunosorbent assay (ELISA) signal when coated particles were probed with polyclonal antibodies against TMV (FIG. 18). These data suggest that cold-temperature ultracentrifugation is an effective method to remove F127 from CPMV particles, but is not as effective for TMV.

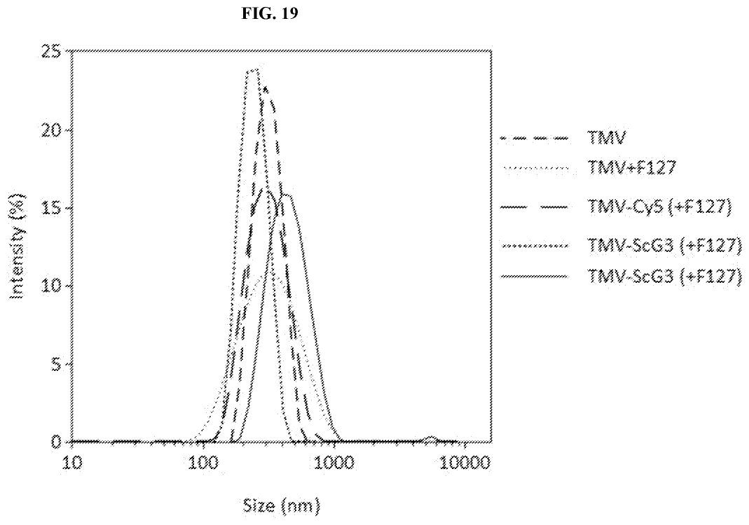

The size, charge, and multivalent display density of conjugated ligands can all play a role in the steric and electrostatic surface environment of plant VNPs, and thus influence the F127 coating process. Indeed, Applicant observed more efficient F127 removal from chemically conjugated TMV compared to native TMV and its conjugates (FIG. 19). Interactions between the terminal poly(ethylene oxide) groups of F127 micelles and the coat protein surface are thought to control F127 coating by physisorption, and the multivalent display of ligands on TMV may perturb these interactions thus reducing the ‘holding power’ of F127, resulting in narrower peak widths for TMV conjugates compared to native TMV. It has been reported that α-Cyclodextrin forms strong interactions with poly(ethylene oxide) units of F127 in solution and prohibits adsorption of F127 on the TMV surface.²⁴ Therefore, further optimized purification methods for F127 removal from TMV could be developed which incorporate an α-Cyclodextrin incubation step to samples just prior to ultracentrifugation purification. Increasing the number of centrifugation purification steps may also improve F127 removal.⁴⁵

### Small-Molecule Bioconjugation to Plant VNPs Using the COAT Strategy

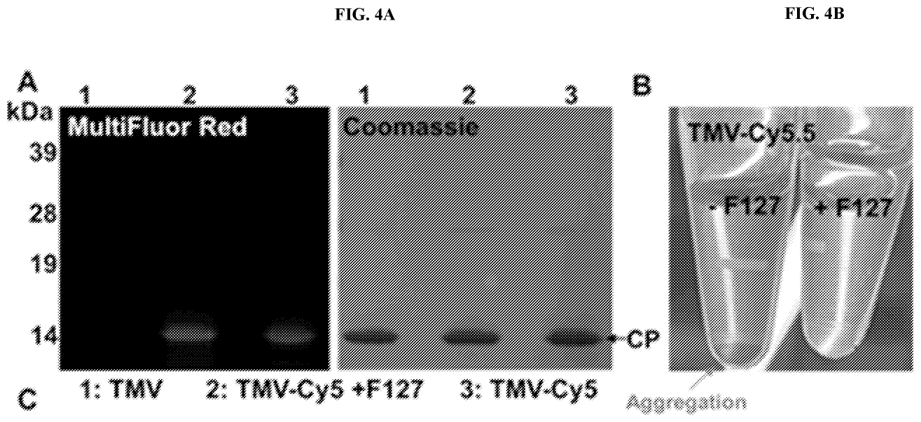

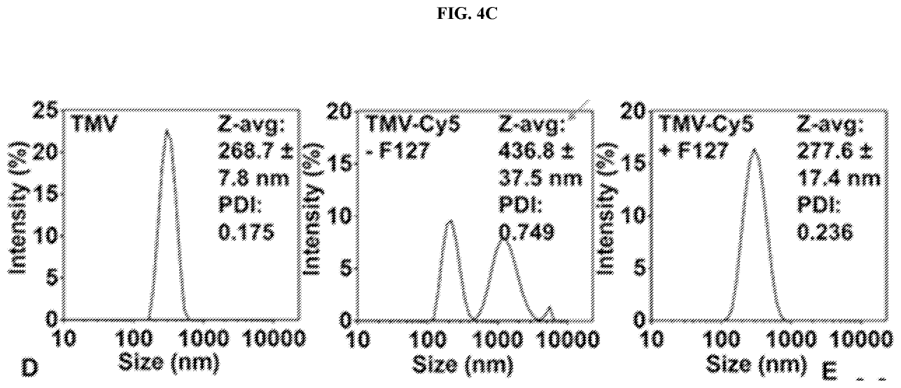

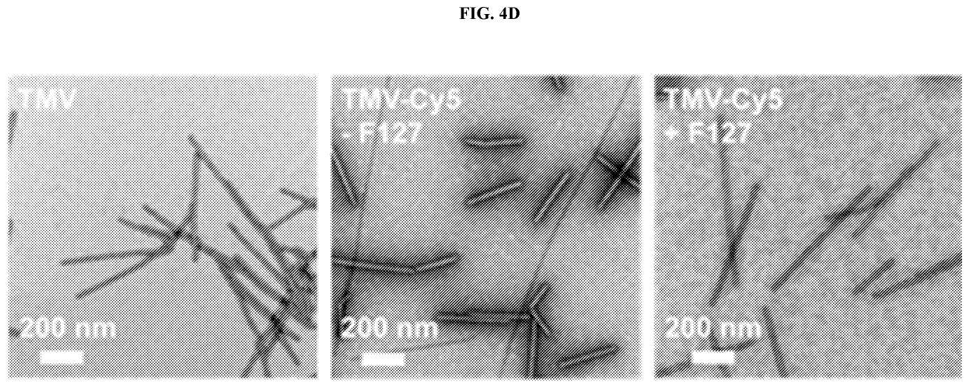

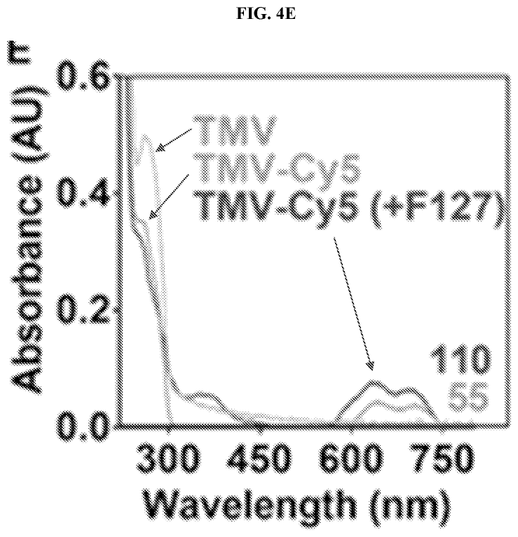

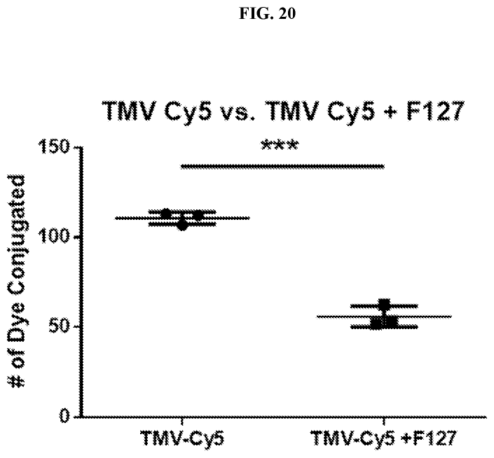

For the conjugation of Cy5.5 to TMV (FIG. 3A), TMV samples coated with F127 at 28 molar equivalents per CP (and uncoated controls) were incubated with the hydrophobic Cy5.5-NHS ligand at 2 molar equivalents per CP. TMV-Cy5.5 conjugates were purified by low-temperature ultracentrifugation to remove excess dye and F127, and were then characterized by SDS-PAGE, DLS, and TEM (FIG. 3). Conjugation of Cy5.5 to both coated and untreated particles was confirmed by SDS-PAGE, as evidenced by the co-colocalization of the Cy5.5 signal and TMV CP band (FIG. 4A). However, the uncoated Cy5.5-conjugated particles were prone to aggregation, revealed by the cloudy solution (data not shown) and high sedimentation velocity, resulting in an observable pellet (FIG. 4B). In contrast, the samples prepared using the COAT method were dispersed and soluble, with no visible signs of aggregation (FIG. 4B). DLS data were consistent with these observations. The Z-average of the TMV-Cy5.5 (+F127) particles was 277.6 nm (PDI=0.236) which is similar to the unconjugated TMV particles (Zₐᵥ=268.7 nm, PDI=0.175) (FIG. 2A). However, the Z-average of the TMV-Cy5.5 (−F127) particles was 436.8 nm (PDI=0.749), resulting in a non-uniform, multimodal intensity profile indicating several different populations of particle sizes (FIG. 4C). TEM imaging confirmed that the COAT method yielded structurally sound TMV-Cy5.5 nanoparticles (FIG. 4D). TMV-Cy5.5(−F127) aggregates were not observed by TEM, but this probably reflects the removal of aggregates during the washing step. Two major populations were formed by the uncoated particles, one of which was more heavily aggregated than the other (as evident by the two DLS populations). Finally, Applicant also evaluated the conjugation efficiency and found that the COAT method doubled the dye conjugation efficiency, resulting in 110 dye molecules conjugated per TMV-Cy5.5 (+F127) particle compared to 55 dye molecules per TMV-Cy5.5(−F127) particle (FIG. 4E). This difference was significant (p<0.001; unpaired two-tailed Student's t-test; FIG. 20).

For the conjugation of Avm to TMGMV (FIG. 3B), alkyne handles were introduced by reacting external tyrosine side chains with diazonium salt (3-ethynyl aniline, 15 molar equivalents per CP) to form a TMGMV-ds intermediate. This was purified by ultracentrifugation to remove excess diazonium salt and resuspended in 0.01 M potassium phosphate buffer (pH 7.4). The COAT procedure was then applied prior to Avm conjugation. TMGMV-ds samples were incubated with the hydrophobic Avm ligand (5 molar equivalents per CP) for 30 min (+F127 particles) or for 3 or 24 h (−F127 particles) and conjugation was carried out using a Cu(I)-catalyzed azide-alkyne cycloaddition (click chemistry) protocol to form the TMGMV-Avm product.

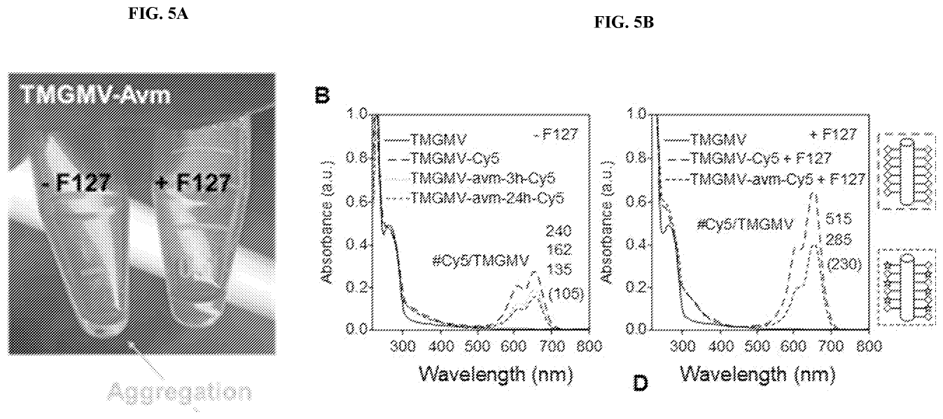

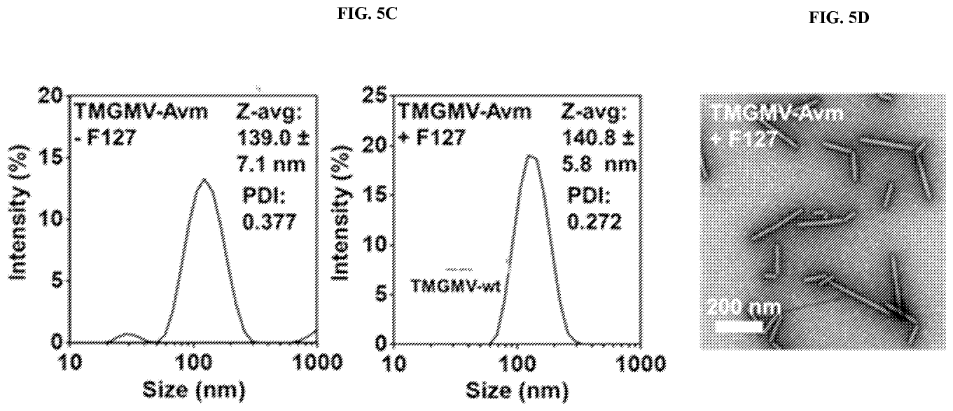

TMGMV-Avm particles were purified by low-temperature ultracentrifugation to remove F127 and excess reagents. The resuspended TMGMV-Avm particles were vortexed and an image was captured using a smartphone camera. The TMGMV-Avm(−F127) particles showed clear evidence of aggregation, resulting in a high sedimentation velocity and an observable pellet (FIG. 5A). In contrast, the TMGMV-Avm(+F127) particles were dispersed and soluble, with no visible signs of aggregation (FIG. 5A). The Z-average of the TMGMV-Avm particles was ˜140 nm for both preparations, but the uncoated samples showed a higher degree of polydispersity (PDI=0.272) compared to the coated particles (PDI=0.084) (FIG. 5C). TEM imaging confirmed that the COAT method yielded structurally sound TMGMV-Avm particles (FIG. 5D). The images also revealed the presence of smaller or broken TMGMV nanoparticles, but this is a common phenomenon for rod-shaped plant viruses and is probably a TEM preparation artifact.

To determine the efficiency of conjugation, Applicant used an indirect fluorescence assay in which TMGMV-ds and TMGMV-Avm particles were reacted for 30 min with sulfo-Cy5.5-azide, and the difference in signal intensity was used to calculate the proportion of unused conjugation handles (FIG. 3B). This revealed that the COAT strategy doubled the conjugation efficiency, resulting in 230 Avm ligands per TMGMV-Avm(+F127) particle compared to 105 ligands per TMGMV-Avm(−F127) particle (FIG. 5B). The coated particles were also more stable. Interestingly, in the same 30-min reaction window, the number of conjugated Cy5 dye molecules was 285 per TMGMV-Avm(+F127) particle but only 135-162 per TMGMV-Avm(−F127) particle (FIG. 5B). This would suggest a lower efficiency of Avm conjugation, but Applicant also observed an increase in Cy5 conjugation for the control TMGMV-ds(+F127) particles (515 dye molecules) compared to the TMGMV-ds(−F127) particles (240 dye molecules). These observations may reflect the presence of a residual F127 coating on the surface of TMGMV-ds control and TMGMV-Avm particles even after ultracentrifugation, and this may enhance the bioconjugation of Cy5 as observed for the corresponding TMV-Cy5 particles (FIG. 3).

### Peptide Bioconjugation to Plant VNPs Using the COAT Strategy

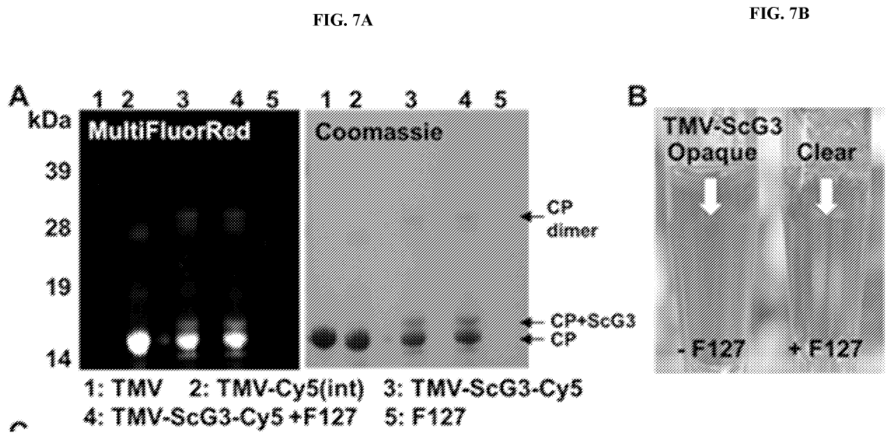

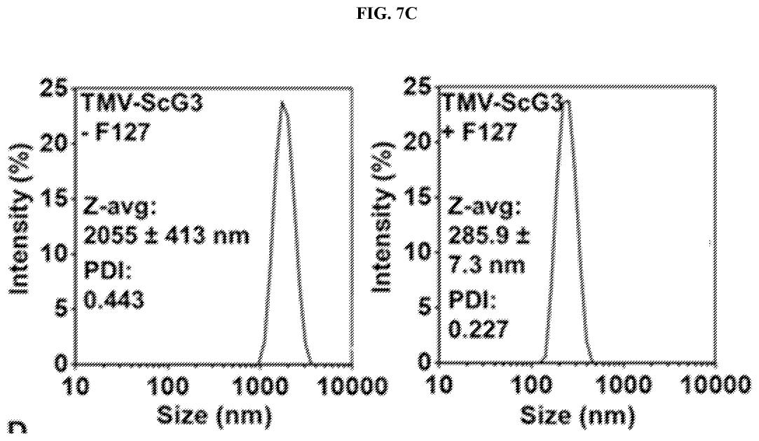

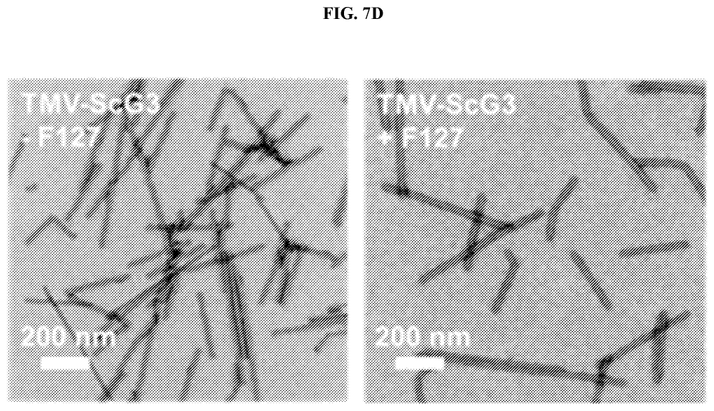

For the conjugation of targeting ligands F3 and ScG3 to TMV, and the therapeutic peptide ApoAI-4FN to CPMV (FIGS. 6A-B), lysine residues on each plant VNP were reacted with the SM(PEG)₄ linker (5 molar equivalents per TMV CP or 3000 molar equivalents per CPMV particle) to form a maleimide intermediate, plant VNP-M(PEG)₄. The COAT procedure was applied before the reaction of TMV with ScG3 or F3 (0.5 molar equivalents per CP) or the reaction of CPMV with ApoAI-4FN (200 molar equivalents per particle). In each case, uncoated particles were conjugated to the same peptides as controls. All samples were purified by low-temperature ultracentrifugation and washing, followed by characterization using the methods described above (FIGS. 7-9).

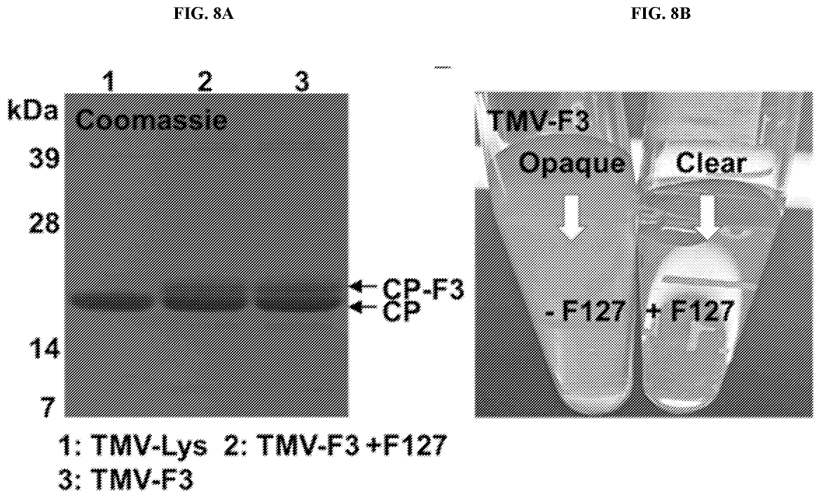

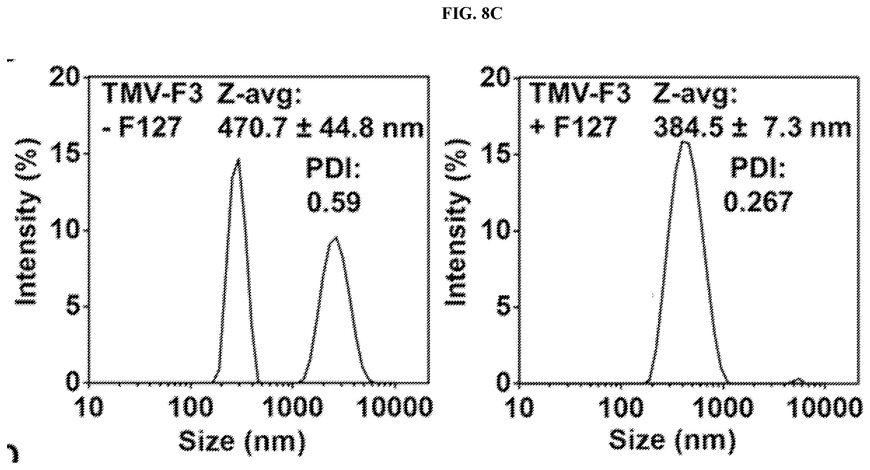

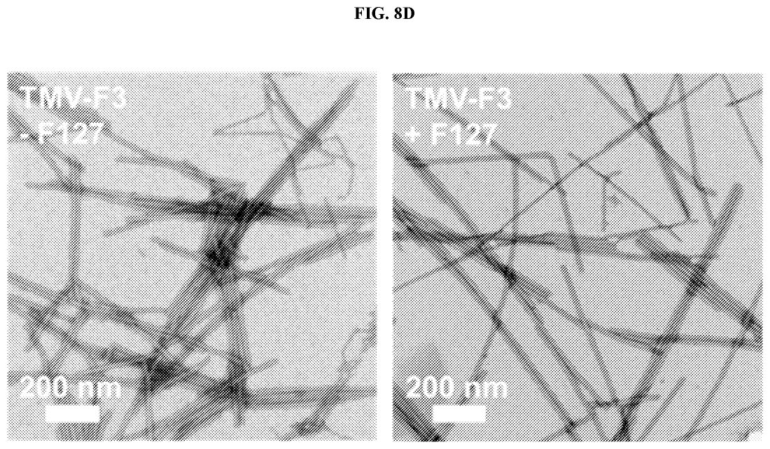

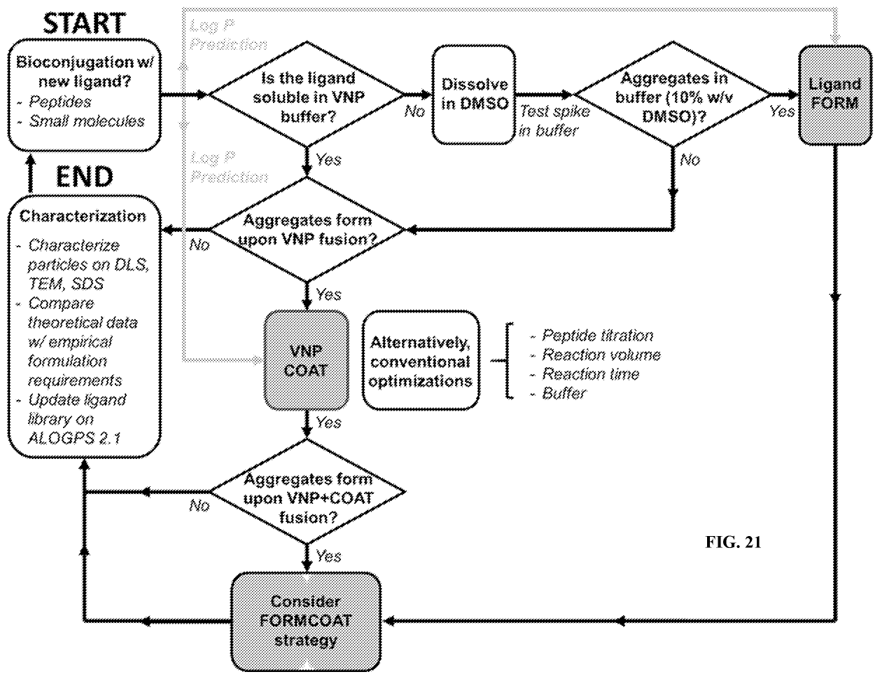

The conjugation of TMV to ScG3 (1.809 kDa) and F3 (3.707 kDa) was confirmed by SDS-PAGE, which revealed the presence of higher-molecular-weight bands at ˜19.3 kDa for CP-ScG3 (FIG. 7A) and 21.2 kDa for CP-F3 (FIG. 8A), in addition to the native TMV CP band at ˜17.5 kDa (FIG. 7A and FIG. 8A). Densitometric analysis indicated ˜25% total CP modification regardless of whether or not the COAT method was used. Therefore, coating with F127 appeared to enhance the conjugation of small-molecule ligands but not peptides. However, the COAT strategy was shown to enhance the stability of plant VNP-peptide conjugates, as shown by the aggregation of the TMV-ScG3 and TMV-F3 samples prepared without F127 but not those prepared with F127 (FIG. 7B and FIG. 8B). DLS confirmed heavy aggregation of the TMV-ScG3(−F127) particles (Zₐᵥ>2000 nm, PDI>0.4) and the TMV-F3(−F127) particles (Zₐᵥ˜470.7 nm, PDI=0.59). In contrast, there was no evidence of aggregation for the TMV-ScG3 (+F127) particles (Zₐᵥ˜285.9 nm, PDI=0.227) (FIG. 7C and FIG. 8C), and although DLS revealed an increase in the hydrodynamic diameter of the TMV-F3 (+F127) particles (Zₐᵥ˜384.5 nm, PDI=0.267), there was no sign of aggregation in the TMV-ScG3 (+F127) or TMV-F3 (+F127) TEM images (FIG. 7D and FIG. 8D).

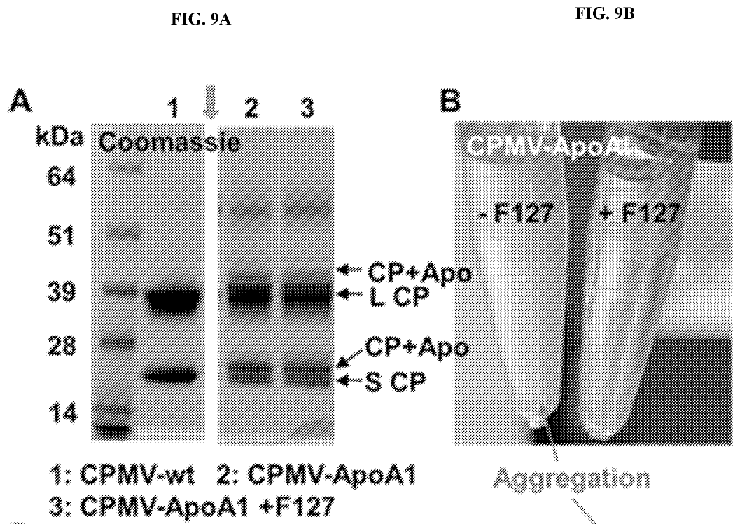

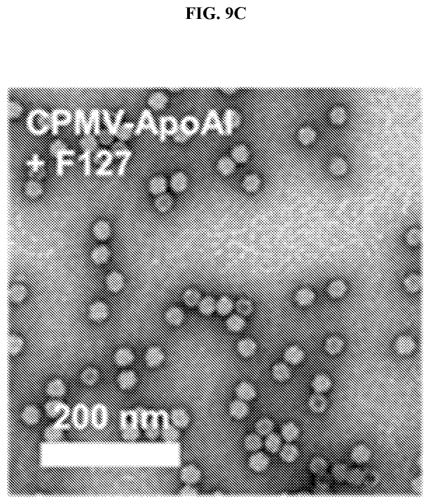

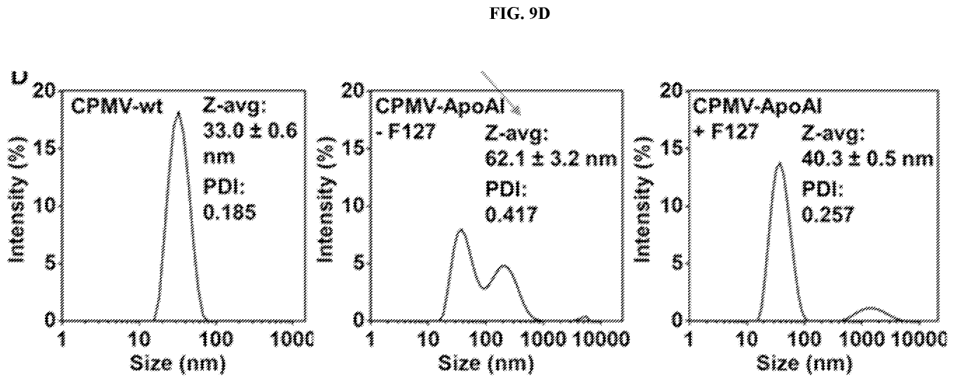

The conjugation of CPMV to ApoAI-4FN (2.6 kDa) was also confirmed by SDS-PAGE, which revealed the presence of native CPMV small(S) and large (L) CP subunits, as well as higher-molecular-weight bands at ˜26.6 kDa (S-CP-4FN) and ˜44.6 kDa (L-CP-4FN) (FIG. 9A). The COAT strategy promoted a small increase in conjugation efficiency (˜59% vs ˜57% for the S-CP, and ˜50% vs ˜43% for the L-CP). The COAT strategy also increased the number of ApoAI-4FN peptides per CPMV from 61 to 66. As observed for the other conjugates, the CPMV-4FN(−F127) particles showed clear visual signs of aggregation whereas the CPMV-4FN(+F127) particles showed good dispersion properties (FIG. 9B). This was supported by the DLS data, which revealed a multimodal intensity profile for the CPMV-4FN(−F127) particles, indicating distinct populations with a Z-average of ˜62.0 nm (PDI=0.417). In contrast, the CPMV-4FN(+F127) particles were characterized by a Z-average of ˜40.3 nm (PDI=0.257), although Applicant detected a low-intensity peak of larger particles contributing to the increase in size, which may indicate the presence of a small fraction of aggregated particles (FIG. 9D). As for the other plant VNP conjugates described above, no aggregates were observed in the TEM images (FIG. 9C).

Having established that the COAT method enhances the stability of TMV and CPMV when conjugated to hydrophobic peptides, Applicant investigated whether this approach was compatible with the conjugation of peptide epitopes used to develop plant VNP-based vaccines.⁴⁶ For the conjugation of peptide epitopes (MyoI and COVID-826) to CPMV (FIGS. 6A-B), lysine residues in the CP were reacted with the SM(PEG)₄ linker (3000 molar equivalents per CPMV particle) to form the maleimide intermediate CPMV-M(PEG) 4. The COAT procedure was applied before the reaction of CPMV with MyoI or COVID-826 (3000 molar equivalents per particle). In each case, uncoated particles were conjugated to the same peptides as controls. Samples were purified by low-temperature ultracentrifugation and washing, followed by characterization using the methods described above.

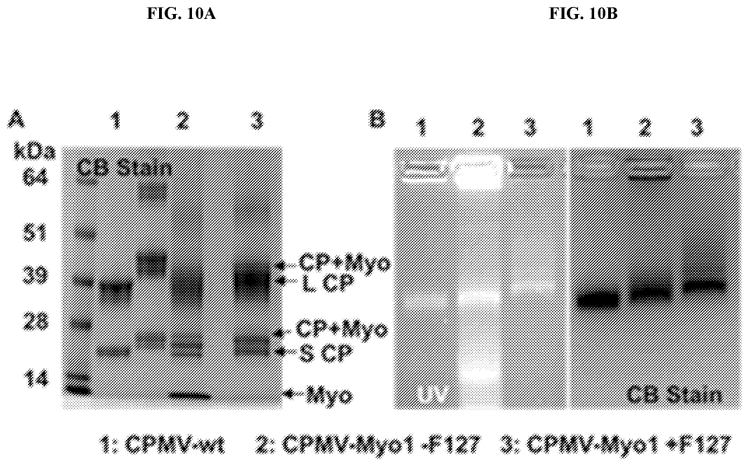

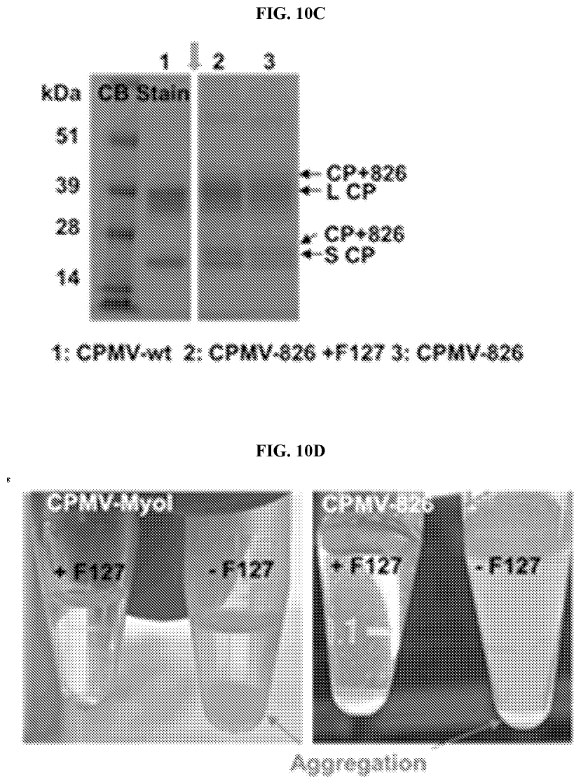

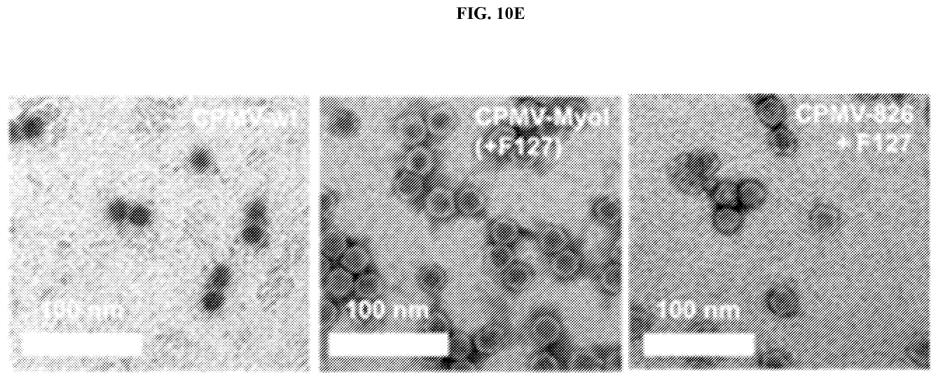

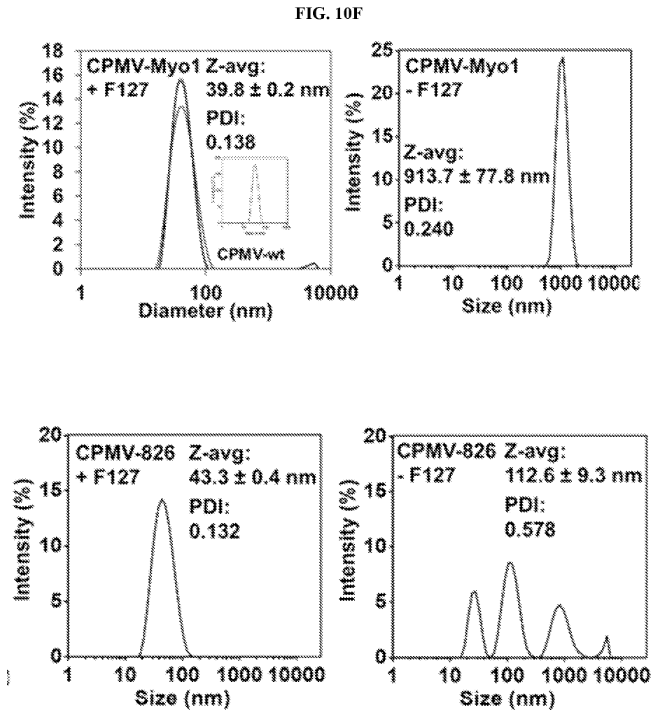

The conjugation of CPMV to MyoI (2.092 kDa) was confirmed by SDS-PAGE, which revealed the presence of native S-CP (˜24 kDa) and L-CP (˜42 kDa) subunits as well as higher-molecular-weight bands at ˜26 kDa (S-CP-MyoI) and ˜44 kDa (L-CP-MyoI) (FIG. 10A). The conjugation of CPMV to COVID-826 was confirmed by the same banding profile (FIG. 10C). There was a significant difference between the coated and uncoated particles revealing a higher conjugation efficiency for both peptides following the COAT procedure. For MyoI, the COAT strategy increased the number of conjugated peptides from 68 (−F127) to 85 (+F127), and for COVID-826, the COAT strategy increased the number of conjugated peptides from 61 (−F127) to 68 (+F127). The analysis of CPMV-MyoI particles indicated ˜60% modification of S-CP and ˜80% modification of L-CP following the COAT process, compared to ˜72% modification of S-CP and ˜41% modification of L-CP without F127. Similarly, the analysis of CPMV-826 particles indicated ˜62% modification of S-CP and ˜50% modification of L-CP following the COAT process, compared to ˜54% modification of S-CP and ˜47% modification for L-CP without F127. In the case of MyoI, the COAT strategy therefore appears to shift the multivalent display tendency from S-CP to L-CP while increasing the overall conjugation efficiency, which should be investigated in more detail. The COAT strategy also enhanced the stability of the plant VNP-peptide conjugates. The aggregation of particles lacking the F127 coat was indicated by the presence of CPMV-MyoI(−F127) particles trapped in the wells of agarose gels leading to the colocalization of the CPMV protein and RNA signals (FIG. 10B) and the formation of visible pellets by both the CPMV-MyoI(−F127) and CPMV-826(−F127) particles (FIG. 10D). Furthermore, SDS-PAGE analysis of the CPMV-MyoI(−F127) particles revealed a band corresponding to the free peptide in lane 2 (FIG. 10A), suggesting the free peptide precipitates in the CPMV-MyoI(−F127) pellet and cannot be purified (FIG. 10D). In contrast, samples prepared using the COAT process were dispersed and soluble with no signs of aggregation. There were also no signs of aggregation in the TEM images (FIG. 10E). DLS confirmed the significant aggregation the CPMV-MyoI(−F127) and CPMV-826(−F127) particles. The Z-average of the CPMV-MyoI(−F127) particles was ˜913.7 nm (PDI=0.240) and that of the CPMV-826(−F127) particles was ˜112.6 nm (PDI=0.578), compared to ˜39.8 nm (PDI=0.138) for the CPMV-MyoI (+F127) particles and ˜43.3 nm (PDI=0.132) for the CPMV-826 (+F127) particles (FIG. 10F). The CPMV-MyoI (+F127) sample included a small proportion of larger particles that were not detected visually in the solution (FIG. 10D) or in the TEM images (FIG. 10E).

### Ligand Formulation (FORMCOAT Strategy)

### General Strategy

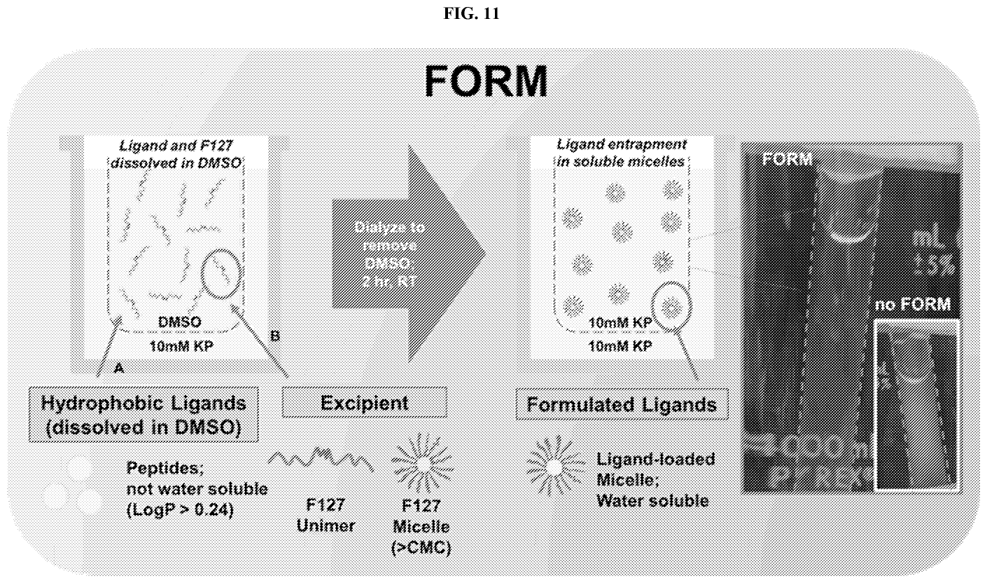

The three remaining SARS-COV-2 epitopes (COVID-106, COVID-153 and COVID-454) as well as the HER2 epitope CH401R required additional F127 preincubation because the peptides were insoluble in the 0.01 M potassium phosphate reaction buffer (pH 7.4). Applicant therefore applied a formulation method adapted from a protocol in which the hydrophobic cyclosporine A peptide (Log P=2.92) was micellized with F127 following dialysis-driven DMSO-aqueous exchange.¹⁸ F127 was used to package the four hydrophobic peptides into micellized, water-soluble formulations (FIG. 11). Accordingly, each peptide was dissolved in DMSO and mixed with 10% (w/w) F127 before dialysis to form peptide-loaded micelles that are stable in aqueous buffer. The formulated peptides (FORM) were then combined with CPMV-MPEG₄ particles that had already been coated with F127 (COAT), in what Applicant describe as the FORMCOAT strategy.

### Peptide Conjugation Using the FORMCOAT Strategy

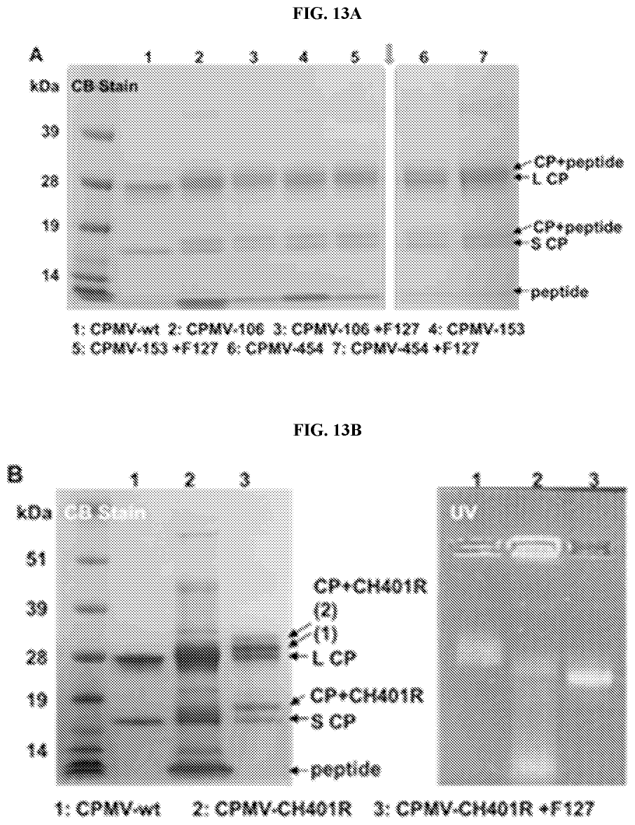

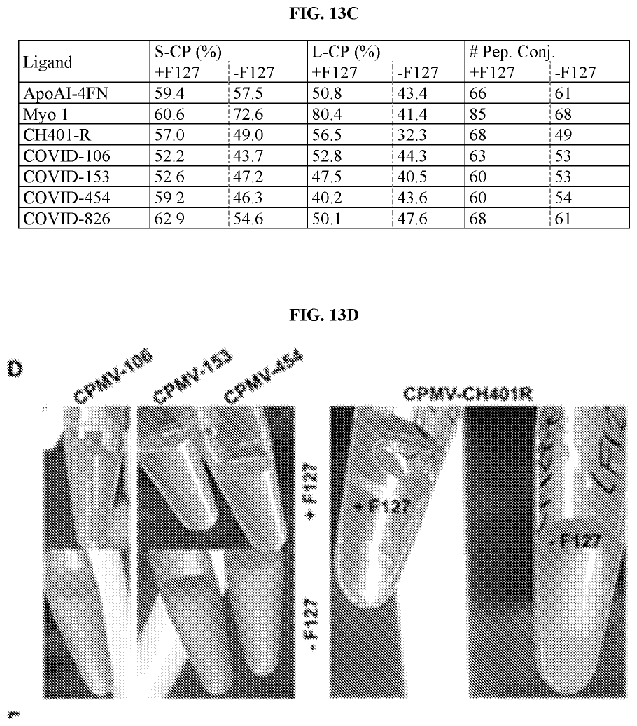

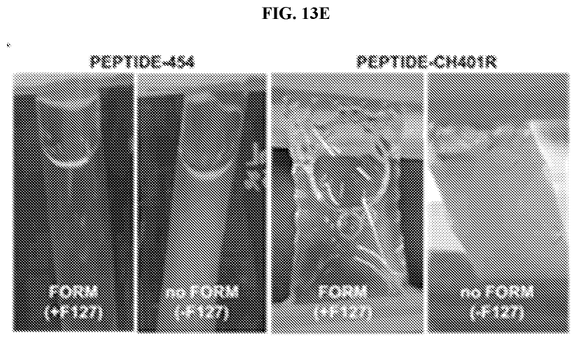

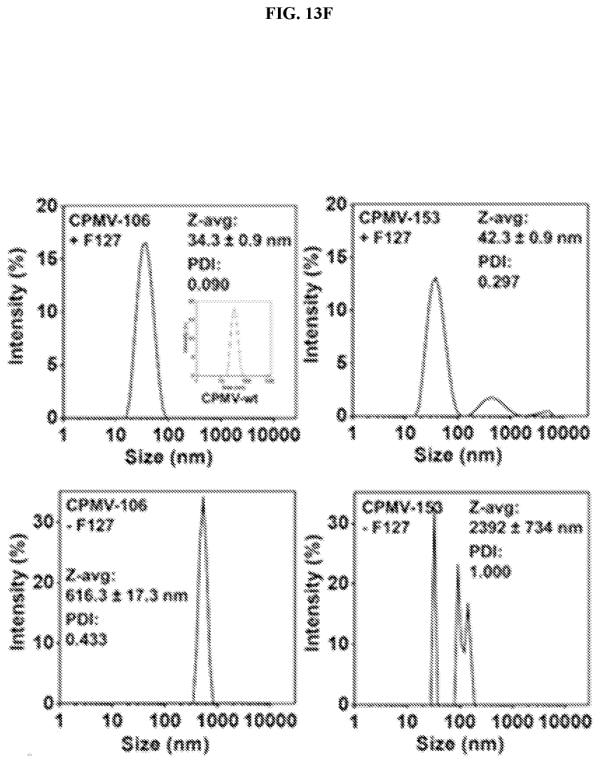

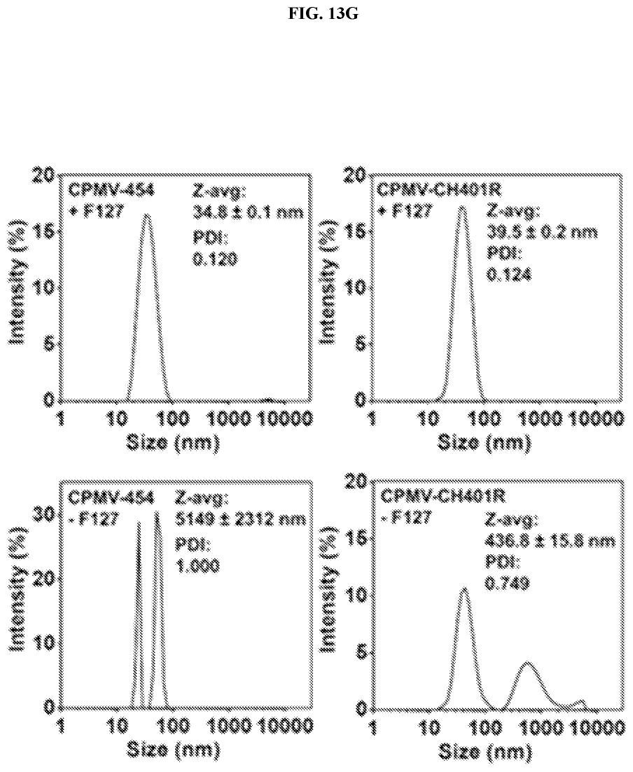

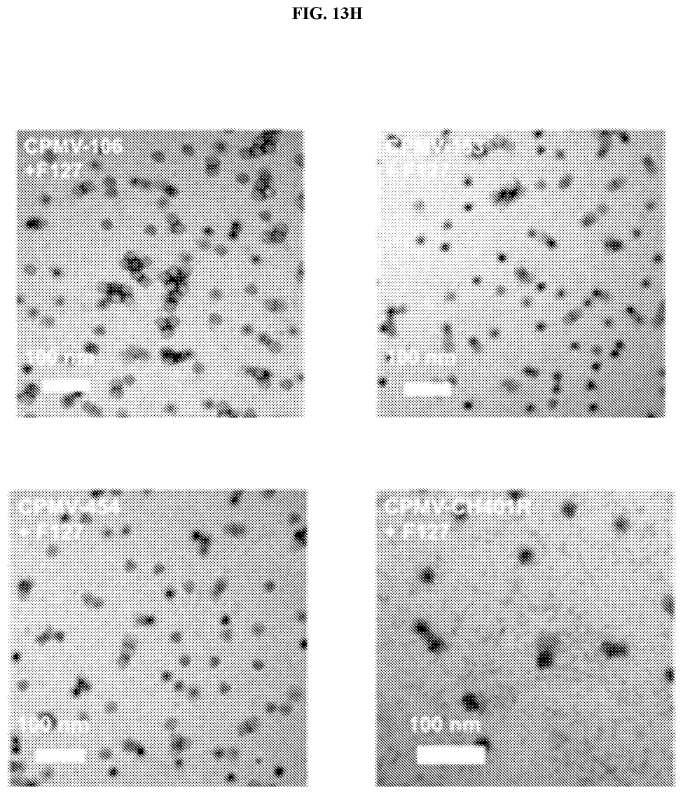

The formulated peptides were mixed with the CPMV-M(PEG)₄ particles coated with F127 at a peptide-dependent molar excess and the reaction mix was purified by low-temperature ultracentrifugation to remove excess peptide and F127. The pellets were washed three times in 0.01 M potassium phosphate buffer (pH 7.4) before characterization using the methods discussed above. In each case, uncoated particles were conjugated to the same peptides as controls. The conjugation of CPMV to peptides CH401R (2.922 kDa), COVID-106 (2.043 kDa), COVID-153 (2.158 kDa), and COVID-454 (2.164 kDa) was confirmed by SDS-PAGE, which revealed the presence of native CPMV S-CP and L-CP subunits as well as higher-molecular-weight bands representing the same subunits displaying each peptide (FIG. 13A, FIG. 13B). Densitometric analysis using ImageJ (FIG. 13C) confirmed that the FORMCOAT method enhanced conjugation efficiency by 11-40% compared to the samples lacking F127, thus increasing the number of peptide ligands per plant VNP. Furthermore, conjugates prepared using the FORMCOAT method were stable and soluble, without visible aggregation (FIG. 13D, FIG. 13E), whereas control samples lacking F127 showed visible signs of aggregation due to the formation of insoluble conjugates. SDS-PAGE also revealed bands at the bottom of the gel, indicating that the insoluble peptides were not removed by purification (FIG. 13A lanes 2 and 4; FIG. 13B lane 2). DLS confirmed that the polydispersity of CPMV conjugates prepared using the FORMCOAT strategy was lower (PDI<0.120) than the corresponding samples lacking an F127 coat (PDI>0.433) and the intensity peak profile was similar to that of native CPMV (FIGS. 13F-13G). The Z-average of the FORMCOAT CPMV-peptide particles ranged from ˜34 to ˜42 nm, whereas the corresponding values for the particles lacking F127 ranged from ˜436 to ˜5149 nm. The CPMV-153 FORMCOAT particles displayed some trace aggregates. However, the samples lacking F127 featured irregular peak shapes unsuitable for measurement, probably reflecting the presence of complex, heterogeneous precipitates of plant VNP-peptide conjugates and the free peptide, as indicated by SDS-PAGE analysis. TEM images confirmed the integrity of the CPMV-peptide conjugates generated using the FORMCOAT strategy (FIG. 13H).

### Retroanalysis of F127-Mediated Bioconjugation and Chemoinformatic Prediction

Ab initio secondary and tertiary structure prediction tools are particularly useful during the early stage of rapid response initiatives to combat emerging biological threats with undefined structural features. Preliminary nucleotide and peptide sequences allow such tools to generate potentially useful 3D information that can be synergized with log P calculators to improve the approximation of peptide ligand hydrophobicity, which predicts the feasibility of chemical fusion and multivalent display on nanoparticles. The virtual pre-screening of peptide or ligand candidates prior to bioconjugation can save time and resources.

To demonstrate how ligand hydrophobicity calculations could inform peptide-plant virus nanoparticle bioconjugation strategies, Applicant retroactively analyzed 17 unique peptide sequences using the PEP-FOLD3 secondary structure prediction tool in concert with ALOGPS v2.1 freeware to predict Log P values.^{47,48} PDB output files from PEP-FOLD3 were converted to 3D Sybyl mol2 or linear SMILES format using Open Babel v2.4.0 freeware for corresponding log P predictions (Table 2, Table 3, Table 4 provides a summary of DLS data comparing the FORMCOAT method versus conventional bioconjugation).⁴⁹ Accordingly, the peptides were assigned to three categories representing the F127 strategy required for stable conjugation: COAT (blue), FORMCOAT (yellow), and N/A (blank). The N/A category consists of peptides that can be conjugated to CPMV without aggregation using conventional bioconjugation methods.⁵⁰ The log P values generated using this approach fell within the range of values reported in the literature.⁵¹

TABLE 2
Secondary structure peptide ranking by decreasing log P and associated
empirical F127 conditions required for stable plant VNP bioconjugation.

| | | 3DMETHOD | | Iso-electric | Net | Estimated |
|---|---|---|---|---|---|---|
| | MW | Log | Log | point | charge | water |
| Peptide | (Da)\hat{ } | P# | S# | (pH)\hat{ } | @ pH 7\hat{ } | solubility\hat{ } |
| *ApoAI-4FN | 2631 | 1.53 | −5.43 | 6.36 | −0.1 | Good |
| **CH401-Rat | 2922 | 1.19 | −5.11 | 8.54 | 0.9 | Poor |
| *Myostatin 1 | 2092 | 1.06 | 5.18 | 8.96 | 1.2 | Poor |
| **COVID-153 | 2158 | 0.98 | −5.47 | 9.73 | 2 | Poor |
| **COVID-106 | 2043 | 0.84 | −5.21 | 9.12 | 0.9 | Poor |
| *F3 | 3707 | 0.81 | −5.18 | 11.08 | 7.9 | Good |
| ***COVID-636 | 1761 | 0.47 | −4.94 | 7.09 | 0 | Poor |
| **COVID-454 | 2164 | 0.32 | −4.81 | 8.79 | 0.9 | Poor |
| *ScG3 | 1809 | 0.27 | −5.1 | 8.96 | 1 | Poor |
| *COVID-826 | 2380 | 0.27 | −4.70 | 10.08 | 1.9 | Good |
| ***COVID-1159 | 1872 | 0.24 | −5.15 | 7.09 | 0 | Good |
| ***COVID-386 | 2261 | −0.06 | −5.25 | 9 | 1.9 | Poor |
| ***COVID-579 | 2401 | −0.10 | −4.81 | 9.06 | 0.9 | Good |
| ***COVID-469 | 2124 | −0.20 | −5.01 | 10.22 | 1.9 | Good |
| ***COVID-820 | 1990 | −0.39 | −4.15 | 6.26 | −0.1 | Good |
| ***COVID-420 | 1857 | −0.55 | −4.38 | 6.21 | −0.1 | Good |
| ***COVID-564 | 1741 | −0.89 | −5.03 | 9.07 | 0.9 | Good |

*COAT
**FORMCOAT
***N/A
#Calculated using ALOGPS2.1 (log P is the octanol/water partition coefficient, with higher values representing increasing lipophilicity).
\hat{ }Calculated using PepCalc (the isoelectric point is the pH condition at which the net charge of the peptide is zero).

After first ranking the peptides in order of decreasing hydrophobicity (decreasing log P), Applicant then cross-mapped our empirical selection of the FORMCOAT or COAT methods to generate stable CPMV-peptide conjugates, and observed a strong correlation. The data suggested that solubility would be challenging for ligands with calculated log P values >0.24, and this could be addressed using the micelle formulation COAT and FORMCOAT strategy (Table 2). It is important to note that the standard in-house molecule/peptide library from ALOGPS 2.1 was used for log P calculations, but the freeware offers users an option to import custom libraries of compounds to better tailor their predictions. Furthermore, secondary structure prediction software can improve the accuracy of log P prediction, as suggested by the tighter correlation observed between log P values and the F127 formulation method when using 3D models (Table 2) rather than linear models (Table 3). This dataset lies at the intersection of chemoinformatics and empirical measurements, and demonstrates that, without any prior knowledge other than a peptide sequence, it is possible to predict the secondary structure, use this model as input for the Log P calculation, and from there predict whether COAT or FORMCOAT strategy is required for successful plant VNP bioconjugation (log P>0.24). A bioconjugation workflow decision tree incorporating this concept is shown in FIG. 21.

### CONCLUSIONS

Applicant has improved the efficiency and stability of plant VNP bioconjugation to 11 hydrophobic ligands, including four that are insoluble without F127 micellar formulation combined with the coating of plant VNPs with F127. Initially, Applicant used F127-coated plant VNP systems as a temporary scaffold to enable multivalent display of hydrophobic ligands, mitigate aggregation, and generate stable solutions containing plant VNP conjugates (COAT strategy). In parallel, Applicant used F127 to formulate and repackage hydrophobic ligands into water-soluble micelles that can be combined with the F127-coated plant VNPs (FORMCOAT strategy). To the best of Applicant's knowledge, this is the first time that F127 has been used in this manner to facilitate the bioconjugation of a hydrophobic ligand to a hydrophilic nanocarrier, and more specifically, a plant virus nanoparticle. This overcomes a significant barrier caused by the insolubility of inherently hydrophobic peptides, and allows the preparation of pure plant VNP-peptide conjugates without significant aggregation. The improvements in conjugation efficiency and stability will maximize the impact of such conjugates in real-world applications, and will be followed up by analyzing their biological activity. For example, recent COVID-19 study indicates that plant VNP-peptide vaccines generated using F127 methods detailed above are immunogenic in vivo.⁵⁰ Finally, the utilization of chemoinformatic prediction software (particularly log P) will accelerate the development of conjugates by circumventing unnecessary screening steps.

### Materials and Methods

Synthesis and formulation materials. Pluronic F127 was purchased from Sigma-Aldrich (St Louis, MO, USA). DMSO was purchased from VWR International (Radnor, PA, USA). Cy5.5 NHS ester was purchased from Lumiprobe (Hunt Valley, MD, USA). Avm was synthesized in-house (FIG. 15). All peptide ligands were prepared by solid-phase peptide synthesis and purchased from GenScript (Piscataway, NJ, USA). SM(PEG)₄, boric acid, monobasic dihydrogen phosphate, and dibasic monohydrogen phosphate were purchased from Thermo Fisher Scientific (Waltham, MA, USA). CPMV and TMV-Lys (described throughout this article as TMV for simplicity) were produced in plants as previously described^{6,52} and were quantified in plant extracts by UV/Vis spectroscopy (CPMV 8260 nm=8.1 mg⁻¹ mL cm⁻¹; TMV 8260 nm=3 mg⁻¹ mL cm⁻¹. Virus integrity was determined by TEM (see below). TMGMV was purchased from Bioprodex (Gainesville, FL, USA), purified by ultracentrifugation followed by overnight dialysis at 4° C. using a Spectrum Labs 10-kDa dialysis membrane (Thermo Fisher Scientific), and finally quantified by UV/Vis (TMGMV 8260 nm=3.16 mg⁻¹ mL cm⁻¹). CPMV stock was stored in 0.1 M potassium phosphate buffer (pH 7.0), whereas TMV and TMGMV stocks were stored in 0.01 M potassium phosphate buffer (pH 7.4).

Pluronic F127 coating of plant VNPs (COAT strategy). Plant VNPs were mixed with 4% (w/w) F127 in a total volume of 0.5-1 mL, followed by three temperature cycles of 1 min on ice (vortex 10 s) and 5 min at room temperature (vortex 10 s). For TMV, F127 (28 molar equivalents per CP) was applied to 2 mg of native particles before conjugation to Cy5.5 (FIG. 3A), or to the intermediate particle TMV-M(PEG)₄ before conjugation to peptides (FIG. 3B). For TMGMV, F127 (28 molar equivalents per CP) was applied to 2 mg of the intermediate particle TMGMV-ds before conjugation to Avm. For CPMV, F127 (480 molar equivalents per particle) was applied to 2 mg of the intermediate particle CPMV-M(PEG)₄ before conjugation to peptides.

Pluronic F127 formulation of ligands. Hydrophobic ligands insoluble in water were dissolved in anhydrous DMSO (10 mg/mL) and mixed with 10% (w/w) F127. Applicant then dialyzed 1 mL of the resulting solution against 1 L 0.01 M potassium phosphate buffer (pH 7.4) using a 1-kDa dialysis membrane, stirring for 2 h at room temperature. Fresh buffer was provided after 30 and 60 min. Formulated ligands were then combined with coated plant VNPs (FORMCOAT strategy).

Bioconjugation of Cy5.5 NHS ester to TMV lysine residues (native chemical ligation). Cy5.5 NHS ester was mixed with native TMV particles (2 molar equivalents per CP) already coated with F127 using the COAT strategy, and the reaction was allowed to proceed for 30 min at room (FIG. 3A). The TMV-Cy5.5 particles were purified by low-temperature ultracentrifugation at 52,000×g for 1 h on a 30% (w/v) sucrose cushion.

Bioconjugation of Avm to TMGMV tyrosine residues (click-chemistry). A diazonium salt was prepared by reacting 75 μL 3 M sodium nitrite with 25 μL 0.68 M 3-ethylaniline in 400 μL 0.3 M p-toluenesulfonic acid monohydrate for 1 h on ice. Applicant then added 15 molar equivalents of the diazonium salt to a 2 mg/mL final concentration of TMGMV in 10 mM borate buffer (pH 8.8) for 30 min on ice (FIG. 3B). The TMGMV-ds intermediate was purified by low-temperature ultracentrifugation at 52,000×g for 1 h on a 30% w/v sucrose cushion. Particles were resuspended in 0.01 M potassium phosphate buffer (pH 7.4) overnight at 4° C. Applicant then applied the COAT preincubation strategy prior to Avm conjugation (FIG. 3B) with uncoated particles as controls. Coated and uncoated particles were incubated with the Avm ligand (5 molar equivalents per CP), along with 2 mM aminoguanidine, 2 mM L-ascorbic acid sodium salt and 1 mM copper (II) sulfate in 0.01 M potassium phosphate buffer (pH 7.4) for 0.5, 3 or 24 h on ice. The TMGMV-Avm product was purified by low-temperature ultracentrifugation at 52,000×g for 1 h on a 30% w/v sucrose cushion. An indirect fluorescence assay was used to infer the number of Avm ligands conjugated to TMGMV. Briefly, 2 molar equivalents of sulfo-Cy5.5-azide were reacted with TMGMV-ds and TMGMV-Avm for 30 min on ice in 0.01 potassium phosphate buffer. The difference in Cy5.5 loading between the TMGMV and TMGMV-Avm formulations was taken as the number of Avm ligands conjugated to TMGMV.

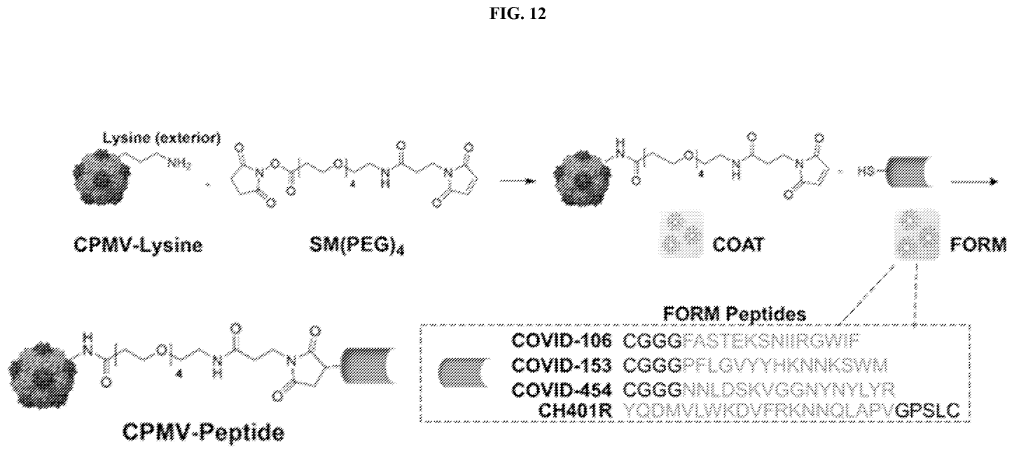

Bioconjugation of peptide ligands to TMV/CPMV lysine residues (maleimide chemistry). External lysine residues were reacted with the SM(PEG)₄ linker at 25 molar equivalents per TMV CP or 5 molar equivalents per CPMV CP (2 mg/mL final plant VNP concentration in 0.01 M potassium phosphate buffer, pH 7.4) for 2 h at room temperature. Plant VNP-M(PEG)₄ intermediates were purified by low-temperature ultracentrifugation at 52,000×g for 1 h on a 30% w/v sucrose cushion. Applicant then applied the COAT procedure, and untreated particles were used as controls. Plant VNP-M(PEG)₄ samples with or without the F127 coating were incubated with hydrophobic peptide ligands, including those formulated with F127 micelles (FIG. 12). For TMV-F3 and TMV-ScG3, the peptide was reacted with TMV-M(PEG)₄ at 0.5 molar equivalents per CP. For CPMV-ApoAI, the peptide was reacted with CPMV-M(PEG)₄ at 200 molar equivalents per particle. For CPMV-MyoI, the SARS-COV-2 peptides, and CPMV-CH401R, the peptides were reacted at 3000 molar equivalents per particle. Reactions with TMV and CPMV were carried out at room temperature and lasted 2 and 12 h, respectively. Conjugates were purified by low-temperature ultracentrifugation at 52,000×g for 1 h on a 30% w/v sucrose cushion. Pellets were washed three times in 0.01 M potassium phosphate buffer (pH 7.4) and were resuspended prior to analysis.

UV/Vis spectroscopy. UV/visible spectra of native and modified CPMV, TMV and TMGMV nanoparticles were recorded using a NanoDrop spectrophotometer (Thermo Fisher Scientific). Samples were dispersed in 0.01 M potassium phosphate buffer (pH 7.4). The number of Cy5 fluorophores per TMGMV CP was determined based on the ratio and the Beer-Lambert law: TMV-Lys & (260 nm)=3.0 mL mg⁻¹ cm⁻¹, molecular weight=39.4×10⁶ g mol⁻¹; CPMV & (260 nm)=8.1 mL mg⁻¹ cm⁻¹, molecular weight=6×10⁶ g mol⁻¹; TMGMV & (260 nm)=3 mL·mg⁻¹ cm⁻¹, molecular weight=39.4×10⁶ g mol⁻¹; Cy5.5 ε (684 nm)=198,000 M⁻¹ cm⁻¹, molecular weight=768 Da.

SDS-PAGE. Applicant denatured 5 μg of plant VNP-peptide conjugates and controls at 100° C. for 5 min in 4×LDS loading dye and 10× reducing solution with a final volume of 20 μL. Denatured plant VNP coat proteins and SeeBlue Plus2 ladder were loaded onto 4-12% or 12% NuPAGE precast gels in 1×3-(N-morpholino) propanesulfonic acid (MOPS) buffer. The samples were separated for 37 min at 200 V and 120 mA. Gels were photographed using the FluorChem R imaging system under white light for Coomassie Brilliant Blue detection and MultiFluor Red light for Cy5.5 detection.

Agarose gel electrophoresis. Applicant loaded 10 μg of CPMV-peptide conjugates and controls onto a 1.2% (w/v) TAE agarose gel and separated the samples at 120 V for 30 min. Gels were photographed using the FluorChem R imaging system.

DLS. The hydrodynamic diameter of native and conjugated plant VNPs was determined by diluting the samples to 1 mg/mL in 0.01 M potassium phosphate buffer (pH 7.4) followed by measurement using a Zetasizer Nano ZSP/Zen5600 instrument (Malvern Panalytical, Malvern, UK). The particle length was calculated as the weighted mean of the intensity distribution.

TEM. TMV samples were diluted to 0.5 mg/mL in Milli-Q water and CPMV samples were diluted to 0.2 mg/mL for optimal sample deposition. Applicant adsorbed 10 μL of each diluted plant VNP sample onto FCF400-CU 400-mesh copper grids (Electron Microscopy Sciences, Hatfield, PA, USA) for 2 min at room temperature, then washed the grid twice for 30 s with Milli-Q water and stained the sample with 10 μL 2% (w/v) uranyl acetate for 1 min. The grid was blotted with Whatman filter paper to remove excess solution and examined at 80 kV using a FEI Company (Hillsboro, OR, USA) Tecnai G2 Spirit transmission electron microscope.

Chemoinformatic prediction. For 3D peptide analysis, the highest probability secondary structure model generated by PEP-FOLD3 was downloaded as a PDB file and converted to Sybyl mol2 format using Open Babel v2.4.0. For linear peptide analysis, PDB output files from PEP-FOLD3 were converted to SMILES format also using Open Babel. For linear small-molecule analysis, CDX files from ChemDraw were converted to SMILES format also using Open Babel. All input files were uploaded into ALOGPS v2.1 with the corresponding input formats to retrieve predicted Log P and Log S values. Peptides and small molecules were considered in separate tables due to the intrinsic differences in ligand size and chemical properties.

Synthesis of azido-avermectin (FIG. 15). Ethyl 5-bromopentanoate (0.100 g, 0.478 mmol, 1 molar equivalent) was reacted with NaN₃ (155 mg, 2.391 mmol, 5 molar equivalents) overnight in DMF (˜10 mL). The crude product was washed three times with water to remove excess NaN₃. The organic layer was dried with Na₂SO₄ and reduced in vacuo to ethyl 5-azidopentanoate (81.9% recovery, 67 mg). Ethyl 5-azidopentanoate (67 mg, 0.392 mmol, 1 molar equivalent) was then reacted with KOH (33 mg, 0.589 mmol, 1.5 molar equivalents) in a 1:1 mixture of methanol (˜5 mL) and water (˜5 mL) overnight. The crude product was diluted with water and acidified with 1 M HCl. The aqueous layer was extracted three times with ethyl acetate. The organic layer was dried with Na₂SO₄ and reduced in vacuo to 5-azidopentanoic acid (63.6% recovery, 35 mg). The 5-azidopentanoic acid (12 mg, 0.086 mmol, 1.5 molar equivalents) and avermectin (50 mg, 0.057 mmol, 1 molar equivalent) were reacted via Steglich esterification consisting of DCC (18 mg, 0.086 mmol, 1.5 molar equivalents) and catalytic DMAP (1 mg, 5% molar fraction) in DCM (˜5 mL) overnight. The reaction was initiated on ice and proceeded as the ice melted. The crude product was placed in the freezer for 30 min and gravity filtered to remove the precipitated DCU byproduct. The remaining product was then extracted with water to remove excess DMAP. The product was then filtered by silica column chromatography with a mobile phase of 4% methanol in DCM. The single and double conjugated azido-avermectin were isolated and reduced in vacuo. Azido-avermectin was dissolved in THE prior to use. The final product (azido-avermectin) and the avermectin starting material were analyzed by ESI-MS (FIGS. 16A-16B).

FT-IR spectroscopy. Applicant loaded 20-μL plant VNP samples onto the Universal ATR Sampling Accessory of a Spectrum Two instrument (Perkin Elmer, Waltham, MA, USA). Spectra were recorded over the range 3500-500 cm⁻¹ with 64 scans (FIG. 13).

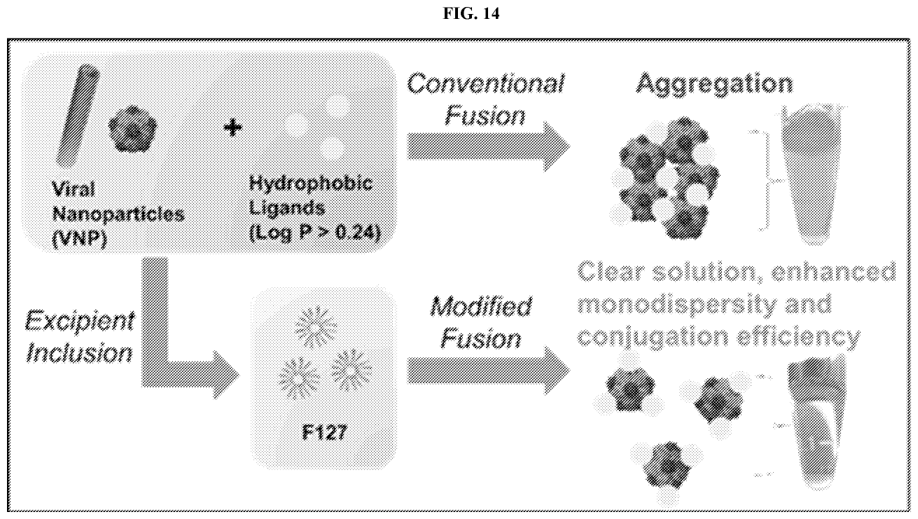

ELISA. COAT-processed plant VNPs were purified by low-temperature ultracentrifugation, washed three times with 0.01 M potassium phosphate buffer, and then coated onto Nunc MaxiSorp 96-well plates overnight at 4° C. Applicant then added 10 μg of CPMV or 100 μg of TMV to each well in a total volume of 100 μL. Applicant washed each well several times with 200 μL PBS containing 0.05% Tween-20 and blocked the wells with 100 μL 3% bovine serum albumin for 1 h. Surface-bound plant VNPs were captured by incubation with polyclonal rabbit anti-TMV (1:100 dilution) or anti-CPMV (1:1000 dilution) antibodies (Pacific Immunology, San Diego, CA, USA) for 1 h on an orbital shaker followed by several washes as above to remove unbound material. Applicant then added a horseradish peroxidase-conjugated goat anti-rabbit secondary antibody (1:5000 dilution) and incubated for 1 h, followed by colormetric development with 3,3′,5,5′-tetramethylbenzidine for 30 min. Absorbance was measured at 450 nm using a Tecan Infinite 200 pro plate reader (FIG. 14).

TABLE 3
Linear peptide ranking by decreasing LogP and associated empirical
F127 conditions required for stable plant VNP bioconjugation.

| | | LINEAR METHOD | | Iso-electric | Net | Estimated |
|---|---|---|---|---|---|---|
| | MW | Log | Log | point | charge@ | Water |
| Peptide | (Da)\hat{ } | P# | S# | (pH)\hat{ } | pH 7\hat{ } | solubility\hat{ } |
| *F₃ | 3707.32 | 1.51 | −4.32 | 11.08 | 7.9 | Good |
| *Myostatin 1 | 2092.38 | −0.4 | −5.15 | 8.96 | 1.2 | Poor |
| **COVID-153 | 2158.46 | −0.5 | −5.4 | 9.73 | 2 | Poor |
| ***COVID-636 | 1760.93 | −0.90 | −4.88 | 7.09 | 0 | Poor |
| *S_{c}G₃ | 1808.97 | −0.95 | −5.11 | 8.96 | 1 | Poor |
| ***COVID-386 | 2261.49 | −1.22 | −5.21 | 9 | 1.9 | Poor |
| *COVID-826 | 2379.73 | −1.23 | −5.08 | 10.08 | 1.9 | Good |
| **CH₄₀₁-Rat | 2922.39 | −1.56 | −4.92 | 8.54 | 0.9 | Poor |
| *ApoAI-₄FN | 2630.92 | −1.83 | −5.35 | 6.36 | −0.1 | Good |
| **COVID-454 | 2164.32 | −2.14 | −4.79 | 8.79 | 0.9 | Poor |
| ***COVID-1159 | 1872.07 | −2.19 | −5.13 | 7.09 | 0 | Good |
| **COVID-106 | 2043.31 | −2.31 | −5.09 | 9.12 | 0.9 | Poor |
| ***COVID-420 | 1857.06 | −2.32 | −4.39 | 6.21 | −0.1 | Good |
| ***COVID-579 | 2400.67 | −2.72 | −4.69 | 9.06 | 0.9 | Good |
| ***COVID-820 | 1990.2 | −2.85 | −4.12 | 6.26 | −0.1 | Good |
| ***COVID-564 | 1740.94 | −2.89 | −4.99 | 9.07 | 0.9 | Good |
| ***COVID-469 | 2124.43 | −3.00 | −4.51 | 10.22 | 1.9 | Good |

*COAT
**FORMCOAT
***N/A
#Calculated using ALOGPS2.1 (log P is the octanol/water partition coefficient, with higher values representing increasing lipophilicity).
\hat{ }Calculated using PepCalc (the isoelectric point is the pH condition at which the net charge of the peptide is zero).

TABLE 4
DLS summary: FORMCOAT method versus conventional bioconjugation

| | | | | | | | # Ligands | |
|---|---|---|---|---|---|---|---|---|
| | | | Conjugate Size (nm) | | Conjugate PDI | | Conjugated | |
| Ligand Type | Ligand | VNP | (+F127) | (−F127) | (+F127) | (−F127) | (+F127) | (−F127) |
| Fluorophore | Cy5.5 NHS | TMV | 277.60 | 436.80 | 0.24 | 0.75 | 110 | 55 |
| Agrochemical | Avermectin-N3 | | 139.00 | 140.80 | 0.27 | 0.38 | 230* | 105* |
| Peptide | F3 | TMV | 384.50 | 470.70 | 0.27 | 0.59 | 533 | 533 |
| | ScG3 | | 285.90 | 2055.00 | 0.23 | 0.44 | 511 | 511 |
| | ApoAI-4FN | CPMV | 40.33 | 112.60 | 0.26 | 0.58 | 66 | 61 |
| | Myostatin 1 | | 39.80 | 913.70 | 0.14 | 0.24 | 85 | 68 |
| | CH401-Rat | | 43.34 | 112.60 | 0.13 | 0.58 | 68 | 49 |
| | COVID-106 | | 34.26 | 616.30 | 0.09 | 0.43 | 63 | 53 |
| | COVID-153 | | 42.27 | 2392.00 | 0.30 | 1.00 | 60 | 53 |
| | COVID-454 | | 34.76 | 5149.00 | 0.12 | 1.00 | 60 | 54 |
| | COVID-826 | | 43.34 | 112.60 | 0.13 | 0.58 | 68 | 61 |

*(+F127) value reflects 30 minute reaction; (−F127) value reflects 24 hour reaction

### EQUIVALENTS

Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control

### REFERENCES

(1) Efthimiadou, E. K.; Theodosiou, M.; Toniolo, G.; Abu-Thabit, N. Y. 15-Stimuli-Responsive Biopolymer Nanocarriers for Drug Delivery Applications. In Stimuli Responsive Polymeric Nanocarriers for Drug Delivery Applications, Volume 1; Makhlouf, A. S. H., Abu-Thabit, N. Y., Eds.; Woodhead Publishing Series in Biomaterials; Woodhead Publishing, 2018; pp 405-432. https://doi.org/10.1016/B978-0-08-101997-9.00019-9.
(2) Estrada, L. P. H.; Champion, J. A. Protein Nanoparticles for Therapeutic Protein Delivery. Biomater. Sci. 2015, 3 (6), 787-799. https://doi.org/10.1039/C5BM00052A.
(3) Chariou, P. L.; Ortega-Rivera, O. A.; Steinmetz, N. F. Nanocarriers for the Delivery of Medical, Veterinary, and Agricultural Active Ingredients. ACS Nano 2020, 14 (3), 2678-2701. https://doi.org/10.1021/acsnano.0c00173.
(4) Veneziano, R.; Moyer, T. J.; Stone, M. B.; Wamhoff, E.-C.; Read, B. J.; Mukherjee, S.; Shepherd, T. R.; Das, J.; Schief, W. R.; Irvine, D. J.; Bathe, M. Role of Nanoscale Antigen Organization on B-Cell Activation Probed Using DNA Origami. Nature Nanotechnology 2020, 15 (8), 716-723. https://doi.org/10.1038/s41565-020-0719-0.
(5) Ma, Y.; Nolte, R. J. M.; Cornelissen, J. J. L. M. Virus-Based Nanocarriers for Drug Delivery. Advanced Drug Delivery Reviews 2012, 64 (9), 811-825. https://doi.org/10.1016/j.addr.2012.01.005.
(6) Geiger, F. C.; Eber, F. J.; Eiben, S.; Mueller, A.; Jeske, H.; Spatz, J. P.; Wege, C. TMV Nanorods with Programmed Longitudinal Domains of Differently Addressable Coat Proteins. Nanoscale 2013, 5 (9), 3808-3816. https://doi.org/10.1039/c3nr33724c.
(7) Chariou, P. L.; Steinmetz, N. F. Delivery of Pesticides to Plant Parasitic Nematodes Using Tobacco Mild Green Mosaic Virus as a Nanocarrier. ACS Nano 2017, 11 (5), 4719-4730. https://doi.org/10.1021/acsnano.7b00823.
(8) Lin, T.; Chen, Z.; Usha, R.; Stauffacher, C. V.; Dai, J.-B.; Schmidt, T.; Johnson, J. E. The Refined Crystal Structure of Cowpea Mosaic Virus at 2.8 Å Resolution. Virology 1999, 265 (1), 20-34. https://doi.org/10.1006/viro.1999.0038.
(9) Hesketh, E. L.; Meshcheriakova, Y.; Thompson, R. F.; Lomonossoff, G. P.; Ranson, N. A. The Structures of a Naturally Empty Cowpea Mosaic Virus Particle and Its Genome-Containing Counterpart by Cryo-Electron Microscopy. Scientific Reports 2017, 7 (1), 539. https://doi.org/10.1038/s41598-017-00533-w.
(10) Schlick, T. L.; Ding, Z.; Kovacs, E. W.; Francis, M. B. Dual-Surface Modification of the Tobacco Mosaic Virus. J. Am. Chem. Soc. 2005, 127 (11), 3718-3723. https://doi.org/10.1021/ja046239n.
(11) Wang, Q.; Kaltgrad, E.; Lin, T.; Johnson, J. E.; Finn, M. G. Natural Supramolecular Building Blocks: Wild-Type Cowpea Mosaic Virus. Chemistry & Biology 2002, 9 (7), 805-811. https://doi.org/10.1016/S1074-5521 (02) 00165-5.
(12) Abrahamian, P.; Hammond, R. W.; Hammond, J. Plant Virus-Derived Vectors: Applications in Agricultural and Medical Biotechnology. Annual Review of Virology 2020, 7 (1), 513-535. https://doi.org/10.1146/annurev-virology-010720-054958.
(13) Bamogo, P. K. A.; Brugidou, C.; Seremé, D.; Tiendrébéogo, F.; Djigma, F. W.; Simpore, J.; Lacombe, S. Virus-Based Pharmaceutical Production in Plants: An Opportunity to Reduce Health Problems in Africa. Virology Journal 2019, 16 (1), 167. https://doi.org/10.1186/s12985-019-1263-0.
(14) Chung, Y. H.; Cai, H.; Steinmetz, N. F. Viral Nanoparticles for Drug Delivery, Imaging, Immunotherapy, and Theranostic Applications. Advanced Drug Delivery Reviews 2020, 156, 214-235. https://doi.org/10.1016/j.addr.2020.06.024.
(15) Wen, A. M.; Steinmetz, N. F. Design of Virus-Based Nanomaterials for Medicine, Biotechnology, and Energy. Chemical Society Reviews 2016, 45 (15), 4074-4126. https://doi.org/10.1039/C5CS00287G.
(16) Zierden, H. C.; Ortiz, J. I.; DeLong, K.; Yu, J.; Li, G.; Dimitrion, P.; Bensouda, S.; Laney, V.; Bailey, A.; Anders, N. M.; Scardina, M.; Mahendroo, M.; Mesiano, S.; Burd, I.; Wagner, G.; Hanes, J.; Ensign, L. M. Enhanced Drug Delivery to the Reproductive Tract Using Nanomedicine Reveals Therapeutic Options for Prevention of Preterm Birth. Science Translational Medicine 2021, 13 (576). https://doi.org/10.1126/scitranslmed.abc6245.
(17) Ensign, L. M.; Tang, B. C.; Wang, Y.-Y.; Tse, T. A.; Hoen, T.; Cone, R.; Hanes, J. Mucus-Penetrating Nanoparticles for Vaginal Drug Delivery Protect Against Herpes Simplex Virus. Science Translational Medicine 2012, 4 (138), 138ra79-138ra79. https://doi.org/10.1126/scitranslmed.3003453.
(18) Kim, Y. C.; Shin, M. D.; Hackett, S. F.; Hsueh, H. T.; Lima e Silva, R.; Date, A.; Han, H.; Kim, B.-J.; Xiao, A.; Kim, Y.; Ogunnaike, L.; Anders, N. M.; Hemingway, A.; He, P.; Jun, A. S.; McDonnell, P. J.; Eberhart, C.; Pitha, I.; Zack, D. J.; Campochiaro, P. A.; Hanes, J.; Ensign, L. M. Gelling Hypotonic Polymer Solution for Extended Topical Drug Delivery to the Eye. Nature Biomedical Engineering 2020, 4 (11), 1053-1062. https://doi.org/10.1038/s41551-020-00606-8.
(19) Schopf, L. R.; Popov, A. M.; Enlow, E. M.; Bourassa, J. L.; Ong, W. Z.; Nowak, P.; Chen, H. Topical Ocular Drug Delivery to the Back of the Eye by Mucus-Penetrating Particles. Trans. Vis. Sci. Tech. 2015, 4 (3), 11-11. https://doi.org/10.1167/tvst.4.3.11.
(20) Yang, M.; Lai, S. K.; Wang, Y.-Y.; Zhong, W.; Happe, C.; Zhang, M.; Fu, J.; Hanes, J. Biodegradable Nanoparticles Composed Entirely of Safe Materials That Rapidly Penetrate Human Mucus. Angew Chem Int Ed Engl 2011, 50 (11), 2597-2600. https://doi.org/10.1002/anie.201006849.
(21) Schneider, C. S.; Xu, Q.; Boylan, N. J.; Chisholm, J.; Tang, B. C.; Schuster, B. S.; Henning, A.; Ensign, L. M.; Lee, E.; Adstamongkonkul, P.; Simons, B. W.; Wang, S.-Y. S.; Gong, X.; Yu, T.; Boyle, M. P.; Suk, J. S.; Hanes, J. Nanoparticles That Do Not Adhere to Mucus Provide Uniform and Long-Lasting Drug Delivery to Airways Following Inhalation. Science Advances 2017, 3 (4), e1601556. https://doi.org/10.1126/sciadv.1601556.
(22) Bodratti, A. M.; Alexandridis, P. Formulation of Poloxamers for Drug Delivery. J Funct Biomater 2018, 9 (1). https://doi.org/10.3390/jfb9010011.
(23) Basak, R.; Bandyopadhyay, R. Encapsulation of Hydrophobic Drugs in Pluronic F127 Micelles: Effects of Drug Hydrophobicity, Solution Temperature, and PH. Langmuir 2013, 29 (13), 4350-4356. https://doi.org/10.1021/la304836e.
(24) Liu, Z.; Gu, J.; Wu, M.; Jiang, S.; Wu, D.; Wang, Q.; Niu, Z.; Huang, Y. Nonionic Block Copolymers Assemble on the Surface of Protein Bionanoparticle. Langmuir 2012, 28 (33), 11957-11961. https://doi.org/10.1021/la302588f.
(25) Surface Layer Modification of Poly(d,l-lactic-co-glycolic acid) Nanoparticles with Targeting Peptide: A Convenient Synthetic Route for Pluronic F127-Tuftsin Conjugate|Bioconjugate Chemistry https://pubs.acs.org/doi/10.1021/acs.bioconjchem.8b00156 (accessed 2021 Sep. 28).
(26) Polymers|Free Full-Text|Pluronic F127-Folate Coated Super Paramagenic Iron Oxide Nanoparticles as Contrast Agent for Cancer Diagnosis in Magnetic Resonance Imaging https://www.mdpi.com/2073-4360/11/4/743 (accessed 2021 Sep. 28).
(27) Nguyen, K. T.; Vinh Le, D.; Ho Do, D.; Huan Le, Q. Development of Chitosan Graft Pluronic® F127 Copolymer Nanoparticles Containing DNA Aptamer for Paclitaxel Delivery to Treat Breast Cancer Cells. Adv. Nat. Sci: Nanosci. Nanotechnol. 2016, 7 (2), 025018. https://doi.org/10.1088/2043-6262/7/2/025018.
(28) Wen, A. M.; Infusino, M.; De Luca, A.; Kernan, D. L.; Czapar, A. E.; Strangi, G.; Steinmetz, N. F. Interface of Physics and Biology: Engineering Virus-Based Nanoparticles for Biophotonics. Bioconjug Chem 2015, 26 (1), 51-62. https://doi.org/10.1021/bc500524f.
(29) Bruckman, M. A.; Czapar, A. E.; VanMeter, A.; Randolph, L. N.; Steinmetz, N. F. Tobacco Mosaic Virus-Based Protein Nanoparticles and Nanorods for Chemotherapy Delivery Targeting Breast Cancer. J Control Release 2016, 231, 103-113. https://doi.org/10.1016/j.jconrel.2016.02.045.
(30) Chariou, P. L.; Dogan, A. B.; Welsh, A. G.; Saidel, G. M.; Baskaran, H.; Steinmetz, N. F. Soil Mobility of Synthetic and Virus-Based Model Nanopesticides. Nature Nanotechnology 2019, 14 (7), 712-718. https://doi.org/10.1038/s41565-019-0453-7.
(31) Cai, H.; Shukla, S.; Wang, C.; Masarapu, H.; Steinmetz, N. F. Heterologous Prime-Boost Enhances the Antitumor Immune Response Elicited by Plant-Virus-Based Cancer Vaccine. J. Am. Chem. Soc. 2019, 141 (16), 6509-6518. https://doi.org/10.1021/jacs.9b01523.
(32) Full article: Effects of maternal immunisation against myostatin on post-natal growth and skeletal muscle mass of offspring in mice https://www.tandfonline.com/doi/full/10.1080/09540100801956596 (accessed 2021 May 22).
(33) Takayama, K.; Noguchi, Y.; Aoki, S.; Takayama, S.; Yoshida, M.; Asari, T.; Yakushiji, F.; Nishimatsu, S.; Ohsawa, Y.; Itoh, F.; Negishi, Y.; Sunada, Y.; Hayashi, Y. Identification of the Minimum Peptide from Mouse Myostatin Prodomain for Human Myostatin Inhibition. J. Med. Chem. 2015, 58 (3), 1544-1549. https://doi.org/10.1021/jm501170d.
(34) Lei, C.; Qian, K.; Li, T.; Zhang, S.; Fu, W.; Ding, M.; Hu, S. Neutralization of SARS-CoV-2 Spike Pseudotyped Virus by Recombinant ACE2-Ig. Nature Communications 2020, 11 (1), 2070. https://doi.org/10.1038/s41467-020-16048-4.
(35) Poh, C. M.; Carissimo, G.; Wang, B.; Amrun, S. N.; Lee, C. Y.-P.; Chee, R. S.-L.; Fong, S.-W.; Yeo, N. K.-W.; Lee, W.-H.; Torres-Ruesta, A.; Leo, Y.-S.; Chen, M. I.-C.; Tan, S.-Y.; Chai, L. Y. A.; Kalimuddin, S.; Kheng, S. S. G.; Thien, S.-Y.; Young, B. E.; Lye, D. C.; Hanson, B. J.; Wang, C.-I.; Renia, L.; Ng, L. F. P. Two Linear Epitopes on the SARS-COV-2 Spike Protein That Elicit Neutralising Antibodies in COVID-19 Patients. Nature Communications 2020, 11 (1), 2806. https://doi.org/10.1038/s41467-020-16638-2.
(36) Wang, H.; Hou, X.; Wu, X.; Liang, T.; Zhang, X.; Wang, D.; Teng, F.; Dai, J.; Duan, H.; Guo, S.; Li, Y.; Yu, X. SARS-COV-2 Proteome Microarray for Mapping COVID-19 Antibody Interactions at Amino Acid Resolution. bioRxiv 2020, 2020.03.26.994756. https://doi.org/10.1101/2020.03.26.994756.
(37) Li, Y.; Lai, D.; Zhang, H.; Jiang, H.; Tian, X.; Ma, M.; Qi, H.; Meng, Q.; Guo, S.; Wu, Y.; Wang, W.; Yang, X.; Shi, D.; Dai, J.; Ying, T.; Zhou, J.; Tao, S. Linear Epitopes of SARS-COV-2 Spike Protein Elicit Neutralizing Antibodies in COVID-19 Patients. Cellular & Molecular Immunology 2020, 17 (10), 1095-1097. https://doi.org/10.1038/s41423-020-00523-5.
(38) Lu, S.; Xie, X.; Zhao, L.; Wang, B.; Zhu, J.; Yang, T.; Yang, G.; Ji, M.; Lv, C.; Xue, J.; Dai, E.; Fu, X.; Liu, D.; Zhang, L.; Hou, S.; Yu, X.; Wang, Y.; Gao, H.; Shi, X.; Ke, C.; Ke, B.; Jiang, C.; Liu, R. The Immunodominant and Neutralization Linear Epitopes for SARS-COV-2. bioRxiv 2020, 2020.08.27.267716. https://doi.org/10.1101/2020.08.27.267716.
(39) Chariou, P. L.; Wang, L.; Desai, C.; Park, J.; Robbins, L. K.; Recum, H. A. von; Ghiladi, R. A.; Steinmetz, N. F. Let There Be Light: Targeted Photodynamic Therapy Using High Aspect Ratio Plant Viral Nanoparticles. Macromolecular Bioscience 2019, 19 (5), 1800407. https://doi.org/10.1002/mabi.201800407.
(40) Park, J.; Gao, H.; Wang, Y.; Hu, H.; Simon, D. I.; Steinmetz, N. F. S100A9-Targeted Tobacco Mosaic Virus Nanoparticles Exhibit High Specificity toward Atherosclerotic Lesions in ApoE−/− Mice. J. Mater. Chem. B 2019, 7 (11), 1842-1846. https://doi.org/10.1039/C8TB02276C.
(41) Sherman, C. B.; Peterson, S. J.; Frishman, W. H. Apolipoprotein A-I Mimetic Peptides: A Potential New Therapy for the Prevention of Atherosclerosis. Cardiology in Review 2010, 18 (3), 141-147. https://doi.org/10.1097/CRD.0b013e3181c4b508.
(42) Singla, P.; Singh, O.; Sharma, S.; Betlem, K.; Aswal, V. K.; Peeters, M.; Mahajan, R. K. Temperature-Dependent Solubilization of the Hydrophobic Antiepileptic Drug Lamotrigine in Different Pluronic Micelles—A Spectroscopic, Heat Transfer Method, Small-Angle Neutron Scattering, Dynamic Light Scattering, and in Vitro Release Study. ACS Omega 2019, 1 (6), 11251-11262. https://doi.org/10.1021/acsomega.9b00939.
(43) Alexandridis, P.; Holzwarth, J. F.; Hatton, T. A. Micellization of Poly(Ethylene Oxide)-Poly(Propylene Oxide)-Poly(Ethylene Oxide) Triblock Copolymers in Aqueous Solutions: Thermodynamics of Copolymer Association. Macromolecules 1994, 27 (9), 2414-2425. https://doi.org/10.1021/ma00087a009.
(44) Zhang, Y.; Song, W.; Geng, J.; Chitgupi, U.; Unsal, H.; Federizon, J.; Rzayev, J.; S ukumaran, D. K.; Alexandridis, P.; Lovell, J. F. Therapeutic Surfactant-Stripped Frozen Micelles. Nature Communications 2016, 7 (1), 11649. https://doi.org/10.1038/ncomms11649.
(45) Sokolsky-Papkov, M.; Kabanov, A. Synthesis of Well-Defined Gold Nanoparticles Using Pluronic: The Role of Radicals and Surfactants in Nanoparticles Formation. Polymers 2019, 11 (10), 1553. https://doi.org/10.3390/polym11101553.
(46) Shin, M. D.; Shukla, S.; Chung, Y. H.; Beiss, V.; Chan, S. K.; Ortega-Rivera, O. A.; Wirth, D. M.; Chen, A.; Sack, M.; Pokorski, J. K.; Steinmetz, N. F. COVID-19 Vaccine Development and a Potential Nanomaterial Path Forward. Nature Nanotechnology 2020, 15 (8), 646-655. https://doi.org/10.1038/s41565-020-0737-y.
(47) Lamiable, A.; Thévenet, P.; Rey, J.; Vavrusa, M.; Derreumaux, P.; Tufféry, P. PEP-FOLD3: Faster de Novo Structure Prediction for Linear Peptides in Solution and in Complex. Nucleic Acids Res 2016, 44 (Web Server issue), W449-W454. https://doi.org/10.1093/nar/gkw329.
(48) Tetko, I. V.; Gasteiger, J.; Todeschini, R.; Mauri, A.; Livingstone, D.; Ertl, P.; Palyulin, V. A.; Radchenko, E. V.; Zefirov, N. S.; Makarenko, A. S.; Tanchuk, V. Yu.; Prokopenko, V. V. Virtual Computational Chemistry Laboratory—Design and Description. J Comput Aided Mol Des 2005, 19 (6), 453-463. https://doi.org/10.1007/s10822-005-8694-y.
(49) O'Boyle, N. M.; Banck, M.; James, C. A.; Morley, C.; Vandermeersch, T.; Hutchison, G. R. Open Babel: An Open Chemical Toolbox. J Cheminform 2011, 3 (1), 33. https://doi.org/10.1186/1758-2946-3-33.
(50) Ortega-Rivera, O. A.; Shin, M. D.; Chen, A.; Beiss, V.; Moreno-Gonzalez, M. A.; Lopez-Ramirez, M. A.; Reynoso, M.; Wang, H.; Hurst, B. L.; Wang, J.; Pokorski, J. K.; Steinmetz, N. F. Trivalent Subunit Vaccine Candidates for COVID-19 and Their Delivery Devices. J. Am. Chem. Soc. 2021, 143 (36), 14748-14765. https://doi.org/10.1021/jacs.1c06600.
(51) Thompson, S. J.; Hattotuwagama, C. K.; Holliday, J. D.; Flower, D. R. On the Hydrophobicity of Peptides: Comparing Empirical Predictions of Peptide Log P Values. Bioinformation 2006, 1 (7), 237-241.
(52) Wen, A. M.; Shukla, S.; Saxena, P.; Aljabali, A. A. A.; Yildiz, I.; Dey, S.; Mealy, J. E.; Yang, A. C.; Evans, D. J.; Lomonossoff, G. P.; Steinmetz, N. F. Interior Engineering of a Viral Nanoparticle and Its Tumor Homing Properties. Biomacromolecules 2012, 13 (12), 3990-4001. https://doi.org/10.1021/bm301278f.
(53) Karolewicz, B.; Górniak, A.; Owczarek, A.; Żurawska-Płaksej, E.; Piwowar, A.; Pluta, J. Thermal, Spectroscopic, and Dissolution Studies of Ketoconazole-Pluronic F127 System. J Therm Anal calorim 2014, 115 (3), 2487-2493. https://doi.org/10.1007/s10973-014-3661-2.

1. A formulation process comprising:
a) admixing a viral nanoparticle (VNP) with an effective amount of about 4 wt % of a poloxamer such as Pluronic F127 (F127) or an equivalent thereof to prepare a first mixture at a temperature of about 25° C.;
b) cooling the mixture conjugate at a temperature below about 19.5° C. to promote the poloxamer such as F127 unimerization and then returning the mixture to a temperature of 25° C. to promote the poloxamer such as F127 micellization; and
c) mixing the mixture prepared by step b).

2. The process of claim 1, wherein one or more of: the effective amount of poloxamer in step a) is from 3.6 wt % to 4.4% wt % and the step a) temperature is from 23.75° C. to 26.25° C., the step b) unimerization temperature is from 18.5° C. to 20.5° C., and the step b) micellization temperature is from 23.75° C. to 26.25° C.

3. The process of claim 1, wherein steps b) and c) are repeated at least twice or at least thrice.

4. The method of claim 1, wherein the effective amount of the poloxamer is from about 15 to about 45 molar equivalents of the poloxamer such as F127 or an equivalent thereof per coat protein (CP) of the VNP, or about from 28 molar equivalents of the poloxamer such as F127 or an equivalent per CP are admixed with the VNP.

5. The method of claim 4, wherein the poloxamer molar equivalent is from 13.5 to 50 molar equivalents or an equivalent per coat protein (CP) of the VNP or from 25 to 31 molar equivalents of the poloxamer or an equivalent per CP.

6. The method of claim 1, wherein the VNP is selected from: tobacco mosaic virus (TMV), cowpea mosaic virus (CPMV) or tobacco mild green mosaic virus (TMGMV), and a modified equivalent thereof, and optionally wherein the modified equivalent is selected from: TMV-lysine, TMV-lysine with a Lys substitution at amino acid position 158, CPMV-lysine, CPMV- with a Lys substitution at amino acid position 158, chemically conjugated TMV, chemically conjugated CPMV, chemically conjugated TMGMV, TMV-Cys5.5, ligand-modified TMV, ligand-modified CPMV, ligand-modified TMGMV, avermectin-TMGMV or TMGMV-Cys5 and further optionally wherein the ligand-modified TMV, ligand-modified CPMV, or ligand-modified TMGMV comprises a ligand selected from a peptide epitope for a vaccine, a peptide epitope for a COVID vaccine, a targeting peptide, an agrochemical, a detectable label, a small molecule, a fluorophore, or a peptide selected from CH401-Rat (SEQ ID NO: 1), Myostatin 1 (SEQ ID NO: 2), COVID-106 (SEQ ID NO: 3), COVID-153 (SEQ ID NO: 4), COVID-454 (SEQ ID NO: 5), COVID-826 (SEQ ID NO: 6), F3 (SEQ ID NO: 7), ScG3 (SEQ ID NO: 8), or ApoAI-4FN (SEQ ID NO: 9).

7.-8. (canceled)

9. The method of claim 6, wherein the ligand is hydrophobic.

10. The method of claim 1, wherein in step a), the VNP and the poloxamer such as F127 or an equivalent are mixed with a hydrophobic Cy5.5-NHS ligand at about 2 molar equivalents per CP, and optionally wherein steps b) and c) are omitted.

11. The method of claim 10, wherein the VNP and the poloxamer are mixed with a Cy5.5-NHS ligand at 1.5 to 2.5 molar equivalents per CP.

12. The method of claim 10, wherein the VNP comprises TMV.

13. The method of claim 1, wherein in step a), the VNP comprises a TMGMV-ds and the poloxamer such as F127 are mixed with avermectin ligand at about 5 molar equivalents per CP, in the presence of Cu(I)-catalyzed azide-alkyne cycloaddition, and optionally wherein steps b) and c) are omitted;
the VNP and poloxamer are mixed with avermectin ligand at from 4.5 to 5.5 molar equivalents per CP; and
the VNP comprises a maleimide-bearing VNP-M(PEG) 4.

14-15. (canceled)

16. The method of claim 1, wherein the VNP comprises maleimide-bearing M(PEG) 4TMV or maleimide-bearing M(PEG) 4 CPMV.

17. The method of claim 1, wherein in step a), the VNP comprises a maleimide-bearing CPMV-M(PEG) 4 mixed with the poloxamer such as F127 or an equivalent thereof and a peptide; optionally wherein the peptide is selected from a COVID19 epitope, a HER2 epitope, cyclosporine A peptide, MyoI, or a peptide selected from CH401-Rat (SEQ ID NO: 1), Myostatin 1 (SEQ ID NO: 2), COVID-106 (SEQ ID NO: 3), COVID-153 (SEQ ID NO: 4), COVID-454 (SEQ ID NO: 5), COVID-826 (SEQ ID NO: 6), F3 (SEQ ID NO: 7), ScG3 (SEQ ID NO: 8), or ApoAI-4FN (SEQ ID NO: 9), and optionally wherein the peptide is selected from a COVID19 epitope, a HER2 epitope, cyclosporine A peptide, MyoI, or a peptide selected from CH401-Rat (SEQ ID NO: 1), Myostatin 1 (SEQ ID NO: 2), COVID-106 (SEQ ID NO: 3), COVID-153 (SEQ ID NO: 4), COVID-454 (SEQ ID NO: 5), COVID-826 (SEQ ID NO: 6), F3 (SEQ ID NO: 7), ScG3 (SEQ ID NO: 8), or ApoAI-4FN (SEQ ID NO: 9) further optionally wherein the ligand is hydrophobic.

18.-19. (canceled)

20. The method of claim 17, wherein the ligand comprises a peptide that comprises a Cys-terminal or N-terminal GGSC (SEQ ID NO: 10) or GGGC linker (SEQ ID NO: 11).

21. The method of claim 20, wherein the GGSC (SEQ ID NO: 10) or GGGC (SEQ ID NO: 11) linker on the peptide reacts with a heterobifunctional maleimide linker SM(PEG)₄.

22. A formulation process for a hydrophobic peptide comprising:
a) dialyzing a water-insoluble peptide dissolved in DMSO and about 10 wt % of a poloxamer such as F127 or an equivalent thereof to form a peptide-loaded micelle (a FORM peptides);
b) mixing the FORM peptide with an M(PEG) 4-VNP with an effective amount of about 4 wt % of a poloxamer such as Pluronic F127 (F127) or an equivalent thereof to prepare a first mixture at a temperature of about 25° C.;
c) cooling the mixture conjugate at a temperature below about 19.5° C. to promote the poloxamer such as F127 unimerization and then returning the mixture to a temperature of about 25° C. to promote the poloxamer such as F127 micellization; and
d) mixing the mixture prepared by step c).

23-27. (canceled)

28. A composition comprising the formulation of claim 1 and a carrier, optionally wherein the carrier is a pharmaceutically acceptable carrier.

29. (canceled)

30. A delivery method comprising administering to a subject in need thereof the composition of claim 28.

31-32. (canceled)

33. A kit comprising the composition of claim 28 and instruction for use.

34. (canceled)

35. A method of promoting cholesterol efflux from a foam cell or inhibiting the growth of a cancer cell comprising contacting the cell with an effective amount of the composition of claim 28 wherein the ligand-modified TMV, ligand-modified CPMV, or ligand-modified TMGMV is modified with the ligand ApoAI-4FN (SEQ ID NO: 9).

36-37. (canceled)
